(19) United States
(12) Patent Application Publication
(10) Pub. No.: US 2026/0242722 A1
(43) Pub. Date: Aug. 20, 2026

(54) FREEZE-DRIED CELL CULTURE MEDIA AND SUPPLEMENTS

(71) Applicant: LIFE TECHNOLOGIES CORPORATION, Carlsbad, CA (US)

(72) Inventors: Joseph A. White, Williamsville, NY (US); Michael Alello, Buffalo, NY (US); Matthew Smonskey, Orchard Park, NY (US); Thomas Mellody, Snyder, NY (US)

(21) Appl. No.: 19/642,092

(22) Filed: Apr. 8, 2026

Related U.S. Application Data
(63) Continuation of application No. PCT/US2024/053976, filed on Oct. 31, 2024.
(60) Provisional application No. 63/594,674, filed on Oct. 31, 2023.

(51) Int. Cl. C12N 5/00

(52) U.S. Cl. CPC C12N 5/0043; C12N 5/005; C12N 2500/32; C12N 2500/34; C12N 2500/38

(57) ABSTRACT

The present disclosure provides freeze-dried eukaryotic cell culture media, feeds, and supplement formulations that have enhanced stability when stored refrigerated or at room temperature. The present disclosure contemplates freeze-dried eukaryotic cell culture media and supplement compositions having reduced moisture content and enhanced stability when compared to non-freeze-dried eukaryotic cell culture media, feed, and supplement compositions. Additional aspects include methods of freeze-drying eukaryotic cell culture media and supplement compositions to enhance stability. Further encompassed are methods for stabilizing eukaryotic cell culture media, feed, or supplement compositions by freeze-drying. Still further aspects include freeze-dried eukaryotic cell culture media, feed, and supplement compositions that are agglomerated or powered media compositions.

### CROSS-REFERENCE OF RELATED APPLICATIONS

This application is a continuation of International Application No. PCT/US2024/053976, filed on Oct. 31, 2024, which claims the benefit of priority to U.S. Provisional Application No. U.S. 63/594,674, filed on Oct. 31, 2023, the entire contents of which are incorporated herein by reference.

### TECHNICAL FIELD

Described herein are eukaryotic freeze-dried cell culture media, feed, and supplement formulations that have enhanced stability when stored at refrigerated- and room temperatures. Embodiments described herein encompass eukaryotic freeze-dried cell culture media and supplement compositions having reduced moisture content and enhanced stability as compared to non-freeze-dried eukaryotic cell culture media, feed, or supplement compositions. Additional embodiments include methods of freeze-drying eukaryotic cell culture media or supplement compositions to enhance stability as well as methods for stabilizing eukaryotic cell culture media, feed, or supplement compositions by freeze-drying. In some aspect, the freeze-dried eukaryotic cell culture media, feed, or supplement compositions are agglomerated or powered media compositions.

### BACKGROUND

Cell culture media provide the nutrients necessary to maintain and grow cells in a controlled, artificial, and in vitro environment. Characteristics and compositions of the cell culture media vary depending on the cellular requirements. Important parameters include osmolality, pH, and nutrient formulations.

A typical eukaryotic cell culture media contains ions (e.g., sodium, potassium, calcium, magnesium, chloride, phosphate, sulfate), amino acids, vitamins, trace elements, and sugar sources. Buffers, such as sodium bicarbonate, antibiotics, and a pH indicator (e.g., phenol red) are often added. Serum, such as fetal bovine serum (FBS), is sometimes added to augment cell growth. FBS often contains more than 1000 components, including proteins, electrolytes, lipids, carbohydrates, hormones, enzymes, and other undefined constituents. Advanced granulation technology (AGT) revolutionized media by providing media in an agglomerated format that dissolves easily and does not create potentially hazardous dust.

One issue with prepared dry media formulations is that they can accumulate excess water molecules and moisture which can react with and degrade sensitive components in the formulation. For example, even agglomerated media can contain small amounts of water that can cause degradative reactions.

What is needed are improved eukaryotic cell culture media, feed, and supplement formulations that have reduced moisture content and enhanced stability at refrigerated and room temperatures. An example of such eukaryotic cell culture media, feed, and supplement formulations are mammalian cell culture media, feed, and supplement formulations.

### SUMMARY

One embodiment described herein is a freeze-dried eukaryotic cell culture media, feed or supplement composition having enhanced stability. In one aspect, the composition is stable at a temperature of about 1° C. to about 40° C. In another aspect, the composition is stable at a temperature of about 15° C. to about 37° C. In another aspect, the composition is stable at refrigeration temperature for about 1 day to about 5 years. In another aspect, the composition is stable at room temperature for about 1 day to about 2 years. In another aspect, the composition is subjected to a freezing temperature ranging from about −60° C. to about −5° C. for about 4 hours to about 10 hours. In another aspect, the composition is subjected to a freezing temperature ranging from about −60° C. to about −40° C. for about 4 hours to about 10 hours. In another aspect, the composition is subjected to a drying temperature ranging from about 30° C. to about 70° C. for about 1 hour to about 4 hours. In another aspect, the composition is subjected to a drying temperature ranging from about 40° C. to about 60° C. for about 1 hour to about 4 hours. In another aspect, the composition has a reduced moisture content as compared to a non-freeze-dried cell culture medium. In another aspect, the composition has a moisture content of at least 1% by mass less than the moisture content of a non-freeze-dried cell culture medium. In another aspect, the composition has a moisture content of about 2-2.6% by mass. In another aspect, the composition is a dry powder media composition. In another aspect, the composition is an agglomerated composition. In another aspect, the composition is animal-origin free. In another aspect, the composition is chemically-defined. In another aspect, the composition is protein-free. In another aspect, the composition is serum-free. In another aspect, the composition is a mammalian cell culture media, feed or supplement composition. In another aspect, the composition has an osmolality of about 200 mOsm/kg to about 3000 mOsm/kg upon reconstitution. In another aspect, the composition has a pH of about 6.0 to about 7.5 upon reconstitution. In yet another aspect, the composition has a pH of about 6.5 to about 7.5 upon reconstitution. In another aspect, the composition comprises: one or more amino acids, salts, buffers, trace minerals, lipids, nucleic acids, or proteins; one or more carbohydrates; and one or more vitamins. In another aspect, the carbohydrates comprise one or more of glucose, fructose, or trehalose. In another aspect, the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.

Another embodiment described herein is a kit comprising a freeze-dried eukaryotic cell culture media, feed, or supplement composition, and one or more containers comprising drums, buckets, boxes, bags, pouches, or combinations thereof. In one aspect, the kit optionally further comprises one or more of labels, manufacturing dates, expiration dates, use by dates, packaging, branding, instructions for use, material safety data sheets (MSDS), hazard or warning placards, or product literature.

Another embodiment described herein is a method for freeze-drying a eukaryotic cell culture media, feed, or supplement composition to enhance stability, the method comprising: subjecting the composition to a freezing temperature ranging from about −60° C. to about −5° C.; and subjecting the composition to a drying temperature ranging from about 30° C. to about 70° C.

Another embodiment described herein is a freeze-dried eukaryotic cell culture media, feed, or supplement composition produced by any of the methods described herein.

Another embodiment described herein is a method for stabilizing a eukaryotic cell culture media, feed, or supplement composition by freeze-drying, the method comprising: providing a cell culture media, feed, or supplement composition or component thereof; subjecting the composition or component thereof to a freezing temperature ranging from about −60° C. to about −5° C.; subjecting the composition or component thereof to a drying temperature ranging from about 30° C. to about 70° C.; and packaging the composition or component thereof in a container.

Another embodiment described herein is the use of the freeze-dried eukaryotic cell culture media, feed, or supplement compositions described herein, or the kits described herein for cell culture. In one aspect, the composition is used for culturing a mammalian cell in vitro. In another aspect, the mammalian cell comprises CHO cells or HEK cells.

Other features and advantages of the disclosure will be apparent from the following descriptions of the drawings, detailed description, clauses, claims, and drawings.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-B show sample freeze drying parameters used to freeze dry DYNAMIS AGT medium. FIG. 1A shows a log of the internal temperature (C) throughout the freeze-drying process using a HARVEST RIGHT Home Freeze Dryer. FIG. 1B shows a log of the internal percent humidity throughout the freeze-drying process using a HARVEST RIGHT Home Freeze Dryer.

FIG. 2A-B show the effects of the freeze-drying process on DYNAMIS AGT. FIG. 2A shows the water content (w/w) of freeze-dried vs. control (non-freeze-dried) DYNAMIS AGT as determined using Karl Fischer analysis. Freeze-drying reduced water content in DYNAMIS AGT by ~1% (w/w). FIG. 2B shows the recovery of sensitive components within DYNAMIS AGT after freeze-drying. Percent recovery was determined by normalizing the post-freeze dry weight of specific components to their pre-freeze-dry weight. Freeze drying did not negatively impact the recovery of sensitive medium components.

FIG. 3A-B show the effects of freeze-drying on the stability of DYNAMIS AGT over time. FIG. 3A shows the water content (w/w) of freeze-dried vs. control (non-freeze-dried) DYNAMIS AGT over 20 days when stored at 37° C. as determined using Karl Fischer analysis. Control is the first 4 bars on the figure and freeze-dried is the second 4 bars on the figure. Water content increased slightly over 5 days (likely due to time spent packaging) in both samples. The water content of the freeze-dried samples remained low compared to control samples through day 20. FIG. 3B shows a comparison of the percent recovery of sensitive components between control and freeze-dried DYNAMIS AGT over 20 days when stored at 37° C. Percent recovery was determined by normalizing all components to Do (day zero) levels for each condition. Freeze drying enhanced the stability of all sensitive components compared to control.

### DETAILED DESCRIPTION

### General Overview

The present application relates to freeze-dried formulations of eukaryotic cell culture media, feed, and supplements that have enhanced stability when stored refrigerated or at room temperature or above room temperature. Dry powder cell culture media can accumulate excess moisture, resulting in unwanted reactivity and degradation of sensitive compounds in the media. Therefore, careful storage and packaging procedures that are typically costly and time consuming are necessary to help prevent the increased moisture content and ensure that the integrity of all media components is preserved. However, even with careful storage and packaging conditions throughout the logistical process following manufacturing, excess water molecules are still present in the media. As a solution to this problem, the present disclosure provides compositions and methods that utilize freeze-drying techniques to advantageously extend the shelf-life and stability of eukaryotic cell culture media, feed, and supplements by reducing the moisture content, and thus the reactivity and degradation of sensitive components in the compositions. The presently described freeze-dried compositions and methods achieve formulations of eukaryotic cell culture media, feed, and supplements having enhanced stability over a greater duration of time when stored at refrigeration or at room temperature or above room temperature. In one aspect, storing cell culture media and supplements at room temperature allows for freeing up valuable refrigerator space and reduces energy usage. Moreover, the freeze-dried formulations described herein can be stored anywhere and are easy to use.

### Definitions

Unless otherwise defined, all technical and scientific terms used herein have the same meaning as commonly understood by one of ordinary skill in the art. For example, any nomenclatures used in connection with, and techniques of cell and tissue culture, biochemistry, molecular biology, immunology, microbiology, genetics, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If one or both end points are modified by the term “about,” the range specified is expanded by a variation of up to +10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “eukaryotic” means belonging to the domain of Eukaryota. Eukaryotic organisms have cells that have a nucleus. Animals, plants, fungi, and many unicellular organisms are eukaryotes. The term “eukaryotic cell culture media, feed or supplement composition” refers to a cell culture media, feed or supplement composition that is formulated to feed and/or support the growth of eukaryotic cells in vitro. An example of a eukaryotic cell culture media, feed or supplement composition is a mammalian cell culture media, feed or supplement composition. A mammalian cell culture media, feed or supplement composition is formulated to feed and/or support the growth of mammalian cells in vitro.

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers to a temperature of about 15-30° C., including all integers and endpoints within the specified range. In another aspect, “room temperature” refers to a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ~20° C., ~22° C., ~25° C., or ~27° C., at standard atmospheric pressure. In another aspect, room temperature refers to 26° C. In another aspect, room temperature refers to 25° C. In another aspect, room temperature refers to 24° C. In another aspect, room temperature refers to 23° C. In another aspect, room temperature refers to 22° C. In another aspect, room temperature refers to 21° C. In another aspect, room temperature refers to 20° C.

As used herein, the terms “refrigeration” or “refrigeration temperature” or “refrigerated” refer to the typical temperature of a refrigeration unit or device in a laboratory setting. In one aspect, the refrigeration unit or device is climate controlled to maintain the temperature at a substantially uniform temperature or with a specific range of temperatures. In one aspect, “refrigeration temperature” refers to a temperature of about 1-10° C., including all integers and endpoints within the specified range. In another aspect, “refrigeration temperature” refers to a temperature of about 2-4° C. In another aspect, “refrigeration temperature” refers to a temperature of about 4° C.

As used herein, the terms “stable” or “stability” refer to the ability of a cell culture media, feed, or supplement formulation as described herein to resist the degradation, oxidation, reactivity, or reduction of one or more of its components at refrigerated and/or room temperature.

As used herein, the phrases “room temperature-stable” or “stable at room temperature” refer to the ability of a cell culture media, feed, or supplement formulation as described herein to resist the degradation, oxidation, reactivity, or reduction of one or more of its components over a period of time at room temperature.

As used herein, the phrase “stable . . . over a period of time” refers to the time period over which a cell culture media, feed, or supplement formulation as described herein will resist or be prevented from degradation, oxidation, reactivity, or reduction at refrigerated and/or room temperature. The period of time can be from 1 day to 2 years, including all integers and dimensions of time (days, weeks, months, or years) within the specified range. The media may be stable for over 30 days, 60 days, 90 days, 120 days, 150 days, 180 days, 210 days, 240 days, 270 days, 300 days, 330 days, 360 days, 390 days, 420 days, 450 days, 480 days, 510 days, 540 days, 570 days, 600 days, 630 days, 660 days, 690 days, 720 days, 730 days or greater than 730 days. The media may be stable for over 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, or greater than 24 months. The media may be stable for over 0.1 of a year, 0.25 of a year, 0.5 of a year, 0.75 of a year, 1 year, 1.1 of a year, 1.25 of a year, 1.5 of a year, 1.75 of a year, 2 years, or greater than 2 years.

The term “extended period of time” is meant a period of time longer than that for which the sample (e.g., nutritive medium, medium supplement, medium subgroup, or buffer) is stored. As used herein, an “extended period of time” therefore means about 1-36 months, about 2-30 months, about 3-24 months, about 6-24 months, about 9-18 months, or about 4-12 months, under a given storage condition, which may include storage at temperatures of about −70° C. to about 40° C., about −70° C. to about 25° C., about −70° C. to about −20° C., about −70° C. to about 4° C., about −20° C. to about 37° C., about −20° C. to about 25° C., about −20° C. to about 4° C. about 0° C. to about 37° C., about 0° C. to about 25° C., about 0° C. to about 4° C., about 4° C. to about 37° C., about 4° C. to about 25° C., about 10° C. to about 37° C., about 10° C. to about 25° C., about 20° C. to about 37° C., or about 20° C. to about 25° C. Assays for determining the biological or biochemical activity of pharmaceutical or clinical compositions, cell culture reagents, nutrients, nutritive media, media supplement, media subgroup, or buffers are well known in the art and are familiar to one of ordinary skill.

The term “powder” or “dry powder” or “dry media powder” as used herein refers to a media composition or formulation for cell culture that is present in dry and fine granular form, wherein the gross appearance may be free flowing. The term “base powder” or “dry base powder” as used herein refers to a dry powder composition or formulation before it is combined with other components for cell culture. In one embodiment, basal media made from dry media powder contains amino acids, glucose, and ions (e.g., calcium, magnesium, potassium, sodium, phosphate) essential for cell growth.

The term “agglomerated,” “agglomerated powder′, “Advanced Granulation Technology (AGT),” or “AGT” as used herein refers to granules of a dry media format produced through an advanced manufacturing process. In one embodiment, the granules encompass a complete media formulation including serum-free, protein-free, and/or chemically-defined media. In another aspect, the granules can be prepared using fluid bed technology that produces agglomerated powders or granules having enhanced characteristics (for example, enhanced solubility). In yet another aspect, the process includes suspending powders in an upwardly moving column of air while simultaneously injecting a controlled and defined amount of liquid into the powder stream to produce a moistened state of the powder; mild heat may be then used to dry the material, producing an agglomerated powder or granule. Preparation of agglomerated media, feeds, nutritive powders, supplements, etc., their properties, and methods of preparing auto pH and auto osmolality of agglomerated media, feeds, nutritive powders, supplements, etc. have been described in U.S. Pat. Nos. 6,383,810 and 6,627,426 and U.S. Pat. App. Pub. No. US 2019/0048312 A1, inter alia, each of which is incorporated by reference for teachings related to agglomerated media.

As used herein, the term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, carbohydrates, lipids, nucleic acids, hormones, vitamins, fatty acids, proteins, and the like. Other ingredients that promote or maintain cultivation of cells ex vivo can be selected by those of skill in the art, in accordance with the particular need.

The terms “cell culture” or “culture” as used herein refer to the maintenance of cells in an artificial, e.g., an in vitro environment. It is to be understood, however, that the term “cell culture” is a generic term and may be used to encompass the cultivation not only of individual eukaryotic (e.g., animal (e.g., mammalian), plant and fungal) cells, but also of tissues, organs, organ systems or whole organisms, for which the terms “tissue culture,” “organ culture,” “organ system culture” or “organotypic culture” may be used interchangeably with the term “cell culture.”

The phrases “cell culture medium,” “culture medium,” “medium formulation,” or “medium” (plural “media” in each case) as used herein refer to a nutritive solution that supports the cultivation and/or growth of cells; these phrases may be used interchangeably. A cell culture medium may be a basal medium (a general medium that requires additional ingredients to support cell growth) or a complete medium that has all or almost all components to support cell growth. Cell culture media may be serum-free or protein-free (one or both), animal-free or animal-origin free, chemically-defined, and may or may not require additional components like growth factors, additives, feeds, supplements, for efficient and robust cell performance.

Nutritive media can also be divided into various “subgroups” that can be prepared and used as described herein. Such subgroups can be combined to produce a nutritive medium. For examples of compatible subgroups and related considerations, see U.S. Pat. Nos. 5,474,931 and 5,681,748, which are incorporated by reference herein for such teachings.

The term “combining” as used herein refers to the mixing or admixing of ingredients in a cell culture medium formulation. Combining can occur in liquid or powder form or with one or more powders and one or more liquids. In another example, two or more powdered components may be mixed and then agglomerated to produce a complex mixture such as media, media supplements, media subgroups, or buffers. Combining also includes mixing dry components with liquid components.

The phrases “concentrate” or “concentrated” are used interchangeably and refer to a component or group of components that are at a concentration higher than that desired in the cell culture medium to be supplemented with the concentrate.

The term “contacting” as used herein refers to the placing of cells to be cultivated into a culture vessel with the medium in which the cells are to be cultivated. The term “contacting” encompasses inter alia mixing cells with medium, perfusing cells with medium, pipetting medium onto cells in a culture vessel, and submerging cells in culture medium. “Contacting” or “in contact with each other” as used herein with regard to specific medium formulations refers to the individual components such as base medium, carbohydrates, and vitamins that are in physical contact with one another and permits the components to interact or react with each other. An admix of components would be in contact with one another.

The term “cultivation” as used herein refers the maintenance of cells in an artificial environment under conditions favoring growth, differentiation, or continued viability, in an active or quiescent state, of the cells. Thus, “cultivation” may be used interchangeably with “cell culture” or any of its synonyms described above.

The term “effective amount” or “effective concentration” refers to an amount of an ingredient, which is available for use. One example is the amount of a vitamin in a culture medium, which is available to cells for use in biological processes normally associated with that vitamin. Thus, an effective amount includes the amount of a cell culture ingredient (e.g., a vitamin or sugar) available for a cell to metabolize. An effective amount of an ingredient can be determined, for example, from the knowledge available to one skilled in the art and/or by experimental determination.

A “feed” or “supplement” as used herein refers to a composition when added to cells in standard culture may be beneficial for its maintenance, or expansion, or growth, or viability, or affects its cell performance, or increases culture longevity or maintains cells in a pseudo-stationary phase wherein product expression continues, or results in a significant increase in final product titer. A feed or supplement may be used interchangeably in this disclosure and refers to any liquid formats (including agglomerated formats) of media components comprising one or more amino acids, sugars, vitamins, buffers, and sometimes peptides, hydrolysates, fractions, growth factors, hormones, etc. required to rebalance or replenish or to modulate the growth or performance of a cell in culture, or a cell culture system. A feed or supplement may be distinguished from a cell culture medium in that it is added to a cell culture medium that can culture a cell. As would be understood by one of skill in the art, sometimes a feed/supplement may comprise those amino acids, sugars, vitamins, buffers, etc. required to rebalance or replenish or modulate the growth or performance of a cell in culture, or a cell culture system. A feed or supplement may or may not be concentrated or may be partially concentrated for certain components only.

The terms “freeze-dried” or “freeze-drying” as used herein refer to cell culture media, feed, or supplement compositions or formulations that have been subjected to a freeze-drying or lyophilization process to reduce the moisture content in the medium, feed, or supplement. In one embodiment, the freeze-drying process encompasses both a freezing temperature (e.g., about −60° C. to about −5° C.) and a subsequent drying temperature (e.g., about 30° C. to about 70° C.).

A cell culture medium is composed of many ingredients and these ingredients vary from one culture medium to another. A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic acid. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See Banes et al., Methods for Preparation of Media, Supplements and Substrate for Serum-Free Animal Cell Culture, Alan R. Liss, N.Y. (1984), which is incorporated by reference herein in its entirety. The osmolality and/or pH, however, may differ in a 1× formulation compared to the culture medium, particularly when fewer ingredients are contained in the 1× formulation. The 1× concentration of any component is not necessarily constant across various media formulations. 1× might therefore indicate different concentrations of a single component when referring to different media. However, when used, 1× will indicate a typical working concentration commonly found in the types of media being referenced. A 1× amount is the amount of an ingredient that will result in a 1× concentration for the relevant volume of medium.

A “10× formulation” as used herein refers to a solution wherein each ingredient in that solution is about 10-times more concentrated than the same ingredient in the cell culture medium. For example, a 10× formulation of RPMI-1640 culture medium may contain, among other ingredients, 2.0 g/L L-arginine, 0.5 g/L L-asparagine, and 0.2 g/L L-aspartic acid (cf. the 1× formulation, above). A “10× formulation” may contain a number of additional ingredients at a concentration about 10 times that found in the 1× culture medium. As will be readily apparent, “5× formulation,” “20× formulation,” “25× formulation,” “50× formulation” and “100× formulation” designate solutions that contain ingredients at about 5-, 20-, 25-, 50- or 100-fold concentrations, respectively, as compared to a working 1× cell culture medium. Again, the osmolality and pH of the media formulation and concentrated solution may vary. See U.S. Pat. No. 5,474,931, which is directed to culture media concentrate technology and is incorporated by reference herein for such teachings.

As used herein, “physiologic pH” is greater than about 4 and less than about 9. Other or particular pH values or ranges, e.g., minimum or maximum pHs of greater than 4.2, 4.5, 4.8, 5.0, 5.2, 5.5, 5.7, 5.8, 6.0, 6.2, 6.5, 6.7, 6.8, 7.0, 7.2, 7.4, 7.5, 7.8, 8.0, 8.2, 8.4, 8.5, 8.7, 8.8, etc. or from about 4.0 to about 9.0, from about 4.0 to about 5.0, from about 5.0 to about 6.0, from about 6.0 to about 7.0, from about 8.0 to about 9.0, from about 4.0 to about 6.0, from about 5.0 to about 7.0, from about 6.0 to about 8.0, from about 7.0 to about 9.0, from about 6.0 to about 9.0, or from about 4.0 to about 7.0 may also be used for dissolving supplements. Some supplements, though not preferred, may only be entirely soluble outside these ranges. In some aspect, “physiologic pH” is about pH 7.4±10%.

An “auto-pH” or “auto-pHing” medium, medium supplement, or buffer as described herein is a formulation which has been formulated such that, upon rehydration with a solvent, the resulting medium, medium supplement, or buffer solution is at a desired pH and does not require adjustment of the pH with acid or base prior to use. For example, an auto-pH culture medium that is formulated to be used at pH 7.4 will, upon rehydration with a solvent, be at pH 7.4 and therefore will be ready for immediate use without further adjustment of the pH.

As used herein, a “solvent” is a liquid that dissolves or has dissolved another ingredient of the medium. Solvents may be used in preparing media, in preparing subgroups, supplements or other formulations, and in reconstituting media or diluting a concentrate in preparation for culturing cells. Solvents may be polar, e.g., an aqueous solvent, or non-polar, e.g., an organic solvent. Solvents may be complex, i.e., requiring more than one ingredient to solubilize an ingredient. Complex solvents may be simple mixtures of two liquids such as alcohol and water or may be mixtures of salts or other solids in a liquid. Two, three, four, five, six, or more components may be necessary in some cases to form a soluble mixture. Simple solvents such as mixtures of ethanol or methanol and water are preferred because of their ease of preparation and handling.

### Freeze-Dried Compositions

Cell culture media compositions require careful storage to ensure that the integrity of all components in the dry powder is conserved. Through the process of freeze-drying, the shelf-life of dry powder media compositions can be increased by reducing the water content to significantly lower amounts allowing for preservation of sensitive components due to the reduced presence of water molecules. Freeze-drying of powdered or granulated media compositions and formulations is a technology that was developed to reduce water content, improve the shelf-life, and improve room temperature stability of the disclosed media products. A key advantage of this freeze-drying technology is that freeze-drying dry powder media compositions and formulations allows further reduction of moisture content and improved product stability. The advantage provided is the extended stability of sensitive components that would otherwise interact with and breakdown in the presence of water. When water content is reduced, less reactivity of sensitive compounds is observed in media compositions and formulations. This freeze-drying technology may be applied to create room temperature stable media compositions and formulations that can be stored at refrigerated temperatures for a greater duration of time. Further, room temperature stability reduces cost throughout the logistical process following the manufacturing of dry powder media compositions.

The process of freeze-drying can create stable media compositions and formulations that can be stored at room temperature for a period of time or stored for longer times in refrigeration. The process for freeze-drying dry powder media compositions can involve, for example, placing the powder onto a tray, setting the parameters of the freeze-dryer, inserting the dry powder media into the freeze-dryer, and running the cycle. The freeze-drying process reduces the water content of the already low-moisture dry powder media and prevents the interaction of sensitive compounds and water reducing degradation of the media. This reduced water content allows for the dry powder media to be preserved for greater periods of time without any change in the components of the media, thus advantageously preserving the compositions and formulations.

Some embodiments described herein are freeze-dried compositions for nutritive media, cell culture media, feeds, media supplements, media subgroups, or buffer; methods for producing nutritive media, media supplements, feeds, media subgroups, or buffers; and the media products produced thereby. Nutritive media, media supplements and media subgroups produced as described herein include any media, media supplement or media subgroup (serum-free or serum-containing) which may be used to support the growth of a cell, which may be a bacterial cell, a fungal cell (particularly a yeast cell), a plant cell or an animal cell (particularly an insect cell, a nematode cell or a mammalian cell, most preferably a human cell), any of which may be a somatic cell, a germ cell, a normal cell, a diseased cell, a transformed cell, a mutant cell, a stem cell, a precursor cell or an embryonic cell. Preferred nutritive media include, but are not limited to, cell culture media, most preferably a bacterial cell culture medium, plant cell culture medium, or animal cell culture medium. Preferred media supplements include, but are not limited to, undefined supplements such as extracts of bacterial, animal or plant cells, glands, tissues or organs, particularly bovine pituitary extract, bovine brain extract and chick embryo extract; and biological fluids (particularly animal sera, and most preferably bovine serum, particularly fetal bovine, newborn calf or normal calf serum, horse serum, porcine serum, rat serum, murine serum, rabbit serum, monkey serum, ape serum or human serum, any of which may be fetal serum) and extracts thereof (more preferably serum albumin and most preferably bovine serum albumin or human serum albumin). Medium supplements may also include defined replacements such as StemPro LipoMAX, OptiMAb, Knock-Out Serum Replacement (Gibco, Thermo Fisher Scientific Inc.), and the like, which can be used as substitutes for the undefined media supplements described above. Such supplements may also comprise defined components, including but not limited to, hormones, cytokines, neurotransmitters, lipids, attachment factors, proteins, and the like.

In one embodiment, the media, media supplements, media subgroups, or buffers, comprises dry agglomerated powders of media, media supplements, media subgroups, or buffers. In one aspect described herein, the agglomerated nutritive media, media supplements, media subgroups, and buffers are produced using fluid bed technology to agglomerate the solutions of media, media supplements, media subgroups, or buffers. Fluid bed technology is a process of producing agglomerated powders having altered characteristics (particularly, for example, solubility) from the starting materials. In general applications of the technology, powders are suspended in an upwardly moving column of air while at the same time a controlled and defined amount of liquid is injected into the powder stream to produce a moistened state of the powder; mild heat is then used to dry the material, producing an agglomerated powder. In one aspect, the agglomerated media, media supplements, media subgroups, or buffers is produced using the proprietary Advanced Granulation Technology (AGT dry media format) (Gibco). See Jayme et al., “A Novel Application of Granulation Technology to Improve Physical Properties and Biological Performance of Powdered Serum-Free Culture Media,” In: Shirahata et al. (eds) Animal Cell Technology: Basic & Applied Aspects, Vol. 12 (2002), Springer, Dordrecht. Specific commercially available agglomerated media, supplements, feeds, and additives include CD CHO AGT, CD OptiCHO AGT, CD FortiCHO AGT, DYNAMIS AGT, VP-SFM AGT, OptiPro AGT, CD Hybridoma AGT, CHO CD EfficientFeed A, B, and C AGT, CHO CD EfficientFeed A+, B+, and C+ AGT Nutrient Supplement, and FunctionMAX TiterEnhancer Additive (all from Gibco; the respective product descriptions are each incorporated by reference herein for such teachings).

The formulations and methods described herein can be used to prepare any nutritive media, media supplement, media subgroup, or buffer and stored for an extended period of time without significant loss of biological or biochemical activity.

Any nutritive medium, medium supplement, medium subgroup, or buffer may be prepared by the methods described herein. Particularly preferred nutritive media, media supplements, and media subgroups that may be prepared as described herein include cell culture media, media supplements, and media subgroups that support the growth of animal cells, plant cells, bacterial cells, or yeast cells. Particularly preferred buffers that may be prepared as described herein include balanced salt solutions, which are isotonic for animal cells, plant cells, bacterial cells, or yeast cells.

Examples of animal cell culture media that may be prepared as described herein include, but are not limited to, DMEM, RPMI-1640, MCDB 131, MCDB 153, MDEM, IMDM, MEM, M199, McCoy's 5A, Williams' Media E, Leibovitz's L-15 Medium, Grace's Insect Medium, IPL-41 Insect Medium, TC-100 Insect Medium, Schneider's Drosophila Medium, Wolf & Quimby's Amphibian Culture Medium, F10 Nutrient Mixture, F12 Nutrient Mixture, and cell-specific serum-free media (SFM) such as those designed to support the culture of keratinocytes, endothelial cells, hepatocytes, melanocytes, CHO cells, 293 cells, PerC6, hybridomas, hematopoetic cells, embryonic cells, neural cells etc. Specific chemically-defined media products include CD CHO Medium (Gibco), CD OptiCHO Medium (Gibco), EX-CELL Advanced CHO Medium (Millipore Sigma-Aldrich), HyClone ActiPro (GE Healthcare Life Sciences). Specific feed supplements include CHO CD EfficientFeed A (or B) AGT Nutritional Supplement (Gibco), CD EfficientFeed C AGT Nutrient Supplement (Gibco), EfficientFeed A+ AGT Supplement (Gibco), EfficientFeed B+ AGT Supplement (Gibco), EfficientFeed C+ AGT Supplement (Gibco), Resurge CD1 Supplement (Gibco), HyClone Cell Boost Supplements (various versions) (GE Healthcare Life Sciences), EX-CELL Advanced CHO Feed 1 (Millipore Sigma-Aldrich), et al. Other media, media supplements, and media subgroups suitable for preparation are available commercially. Formulations for these media, media supplements and media subgroups, as well as many other commonly used animal cell culture media, media supplements and media subgroups are known in the art and available from commercial suppliers, e.g., Thermo Fisher Scientific Inc., Life Technologies, Gibco, Invitrogen, et al.

Examples of plant cell culture media that may be prepared as described herein include, but are not limited to, Anderson's Plant Culture Media, CLC Basal Media, Gamborg's Media, Guillard's Marine Plant Culture Media, Provasoli's Marine Media, Kao and Michayluk's Media, Murashige and Skoog Media, Mccown's Woody Plant Media, Knudson Orchid Media, Lindemann Orchid Media, or Vacin and Went Media. Formulations for these media, which are commercially available, as well as for many other commonly used plant cell culture media, are known in the art and available from commercial manufacturers.

Examples of bacterial cell culture media that may be prepared as described herein include, but are not limited to, Trypticase Soy Media, Brain Heart Infusion Media, Yeast Extract Media, Peptone-Yeast Extract Media, Beef Infusion Media, Thioglycollate Media, Indole-Nitrate Media, MR-VP Media, Simmons' Citrate Media, CTA Media, Bile Esculin Media, Bordet-Gengou Media, Charcoal Yeast Extract (CYE) Media, Mannitol-salt Media, MacConkey's Media, Eosin-methylene blue (EMB) media, Thayer-Martin Media, Salmonella-Shigella Media, and Urease Media. Formulations for these media, which are commercially available, as well as for many other commonly used bacterial cell culture media, are well-known in the art and may be found for example in the DIFCO & BBL Manual, 2^{nd} ed. (Becton, Dickinson and Company, 2009) and in the Manual of Clinical Microbiology (American Society for Microbiology, Washington, D.C.).

Examples of fungal cell culture media, particularly yeast cell culture media, which may be prepared as described herein include, but are not limited to, Sabouraud Media and Yeast Morphology Media (YMA). Formulations for these media are commercially available and are known in the art.

One embodiment described herein is a freeze-dried and temperature-stable cell culture media composition, media supplement, media subgroup, or kit thereof. An exemplary media formulation is provided in Table 1. The exemplary media composition can be formulated as a liquid or dry powder that is reconstituted or dissolved in water prior to use. Liquid components, such as fetal bovine serum, are typically added after dissolution of the dry powder. The media can also be prepared as agglomerated granules using fluid bed technology. Dry powder media and agglomerated media can be prepared as described herein.

TABLE 1
Exemplary Media or Supplement Formulations

| | | Exemplary Mass |
|---|---|---|
| Component | Exemplary Species | Percent (%) |
| Carbohydrate(s) | glucose, fructose, trehalose, etc. | 20-65 |
| (carbon source) | | |
| Vitamins | retinol (A), thiamine (B₁), riboflavin | 1-5 |
| | (B₂), niacinamide (B₃), pantothenic | |
| | acid (B5), pyridoxamine (B₆), biotin | |
| | (B₇), folic acid (B₉) cobalamin (B₁₂), | |
| | ascorbic acid (C), cholecalciferol (D), | |
| | tocopherol (E), phylloquinone (K), | |
| | choline, inositol, lipoic acid, para- | |
| | aminobenzoic acid | |
| Amino Acids | alanine, arginine, aspartic acid, | 20-40 |
| | glutamic acid, glycine, histidine, | |
| | isoleucine, methionine, phenylalanine, | |
| | proline, hydroxyproline, serine, | |
| | threonine, tryptophan, valine, | |
| | tyrosine, cysteine, lysine | |
| Inorganic salts, | salts of sodium, potassium, | 2-10 |
| buffers | magnesium, calcium, ammonium, | |
| | phosphate, carbonate, sulfate | |
| Trace minerals | iron, manganese, copper, iodine, zinc, | 0.01-0.05 |
| | cobalt, fluoride, chromium, | |
| | molybdenum, selenium, nickel, | |
| | silicon, vanadium | |
| Additives | antibiotics, pH indicators, surfactants, | 0.1-5 |
| | amines, lipids, etc. | |
| Proteins: Serum or | fetal bovine serum (FBS), serum | 1-10 |
| serum replacement | replacement (e.g., KnockOut, Gibco) | |

The exemplary media or supplement formulation shown in Table 1 can be formulated in various formulations and kits.

Any of the above media, media supplement, media subgroup, or buffer that can be prepared as described herein may also include one or more additional components, such as indicating or selection agents (e.g., dyes, antibiotics, amino acids, enzymes, substrates, and the like), filters (e.g., charcoal), salts, polysaccharides, ions, detergents, stabilizers, and the like. The embodiment is not limited to presently formulated media but is broadly applicable to any media formulation or supplement for culturing cells.

In another embodiment described herein, the culture media may comprise one or more buffer salts, preferably sodium bicarbonate, at concentrations sufficient to provide optimal buffering capacity for the culture medium. In one aspect, the one or more buffer comprises acetic acid, acetylsalicylic acid, adipic acid, alginic acid, ascorbic acid, aspartic acid, benzoic acid, benzenesulfonic acid, bisulfic acid, boric acid, butanoic acid, butyric acid, camphoric acid, camphorsulfonic acid, carbonic acid, citric acid, cyclopentanepropionic acid, digluconic acid, dodecylsulfic acid, ethanesulfonic acid, formic acid, fumaric acid, glyceric acid, glycerophosphoric acid, glycine, gly-glycine, gluco heptanoic acid, gluconic acid, glutamic acid, glutaric acid, glycolic acid, hemisulfic acid, heptanoic acid, hexanoic acid, hippuric acid, hydrobromic acid, hydrochloric acid, hydroiodic acid, hydroxyethanesulfonic acid, lactic acid, maleic acid, malic acid, malonic acid, mandelic acid, methanesulfonic acid, mucic acid, naphthalenesulfonic acid, naphthilic acid, nicotinic acid, nitrous acid, oxalic acid, pelargonic, phosphoric acid, propionic acid, pyruvic acid, saccharin, salicylic acid, sorbic acid, succinic acid, sulfuric acid, tartaric acid, thiocyanic acid, thioglycolic acid, thiosulfuric acid, tosylic acid, undecylenic acid, MES, bis-tris methane, ADA, ACES, bis-tris propane, PIPES, MOPSO, cholamine chloride, MOPS, BES, TES, HEPES, DIPSO, MOBS, acetamido glycine, TAPSO, TEA, POPSO, HEPPSO, EPS, HEPPS, Tricine, Tris(hydroxymethyl)aminomethane (tromethamine), glycinamide, glycylglycine, HEPBS, Bicine, TAPS, AMPB, CHES, AMP, AMPSO, CAPSO, CAPS, CABS, combinations thereof, or salts thereof. In one aspect, the buffer comprises one or more of phosphate, sulfate, carbonate, formate, acetate, propionate, butanoate, lactate, glycine, maleate, pyruvate, citrate, aconitate, isocitrate, α-ketoglutarate, succinate, fumarate, malate, oxaloacetate, aspartate, glutamate, tris(hydroxymethyl)aminomethane (tromethamine), combinations thereof, or salts thereof. In one aspect, the buffer is sodium carbonate or sodium bicarbonate.

According to one aspect described herein, a buffer salt, such as sodium bicarbonate, may be added to the medium prior to, during, or following agglomeration of the medium. In one example of this aspect described herein, the sodium bicarbonate may be added to the culture medium prior to, during or following agglomeration with an appropriate solvent (such as water, serum or a pH-adjusting agent such as an acid (e.g., HCl at a concentration of 1 M to 5 M, 0.1 M to 5 M, or preferably at 1 M) or a base (e.g., NaOH at a concentration of 1 M to 5 M, 0.1 M to 5 M, or preferably at 1 M) such that, upon reconstitution of the agglomerated medium the culture medium is at the optimal or substantially optimal pH for cultivation of a variety of cell types. For example, bacterial cell culture media prepared by the present methods will, upon reconstitution, preferably have a pH of about 4-10, more preferably about 5-9 or about 6-8.5. Fungal (e.g., yeast) cell culture media prepared by the present methods will, upon reconstitution, preferably have a pH of about 3-8, more preferably about 4-8 or about 4-7.5; animal cell culture media prepared by the present methods will, upon reconstitution, preferably have a pH of about 6-8 or about 7-8, more preferably about 7-7.5, or about 7.2-7.4; and plant cell culture media prepared by the present methods will, upon reconstitution, preferably have a pH of about 4-8, preferably about 4.5-7, 5-6 or 5.5-6. Optimal pH for a given culture medium to be used on a particular cell type may also be determined empirically by one of ordinary skill using art-known methods. For example, gastric cells may be cultured at pHs well below those of other cells, for example, pH 1-3. One of ordinary skill appreciates that other cells adapted to harsh environments may have special tolerances or needs that might be outside the normal ranges that satisfy culture conditions for commonly cultured cells.

In another example, one or more buffer salts, e.g., sodium bicarbonate, may be added directly to a nutritive medium. In a related aspect, a pH-adjusting agent such as an acid (e.g., HCl) or a base (e.g., NaOH) may be added to a nutritive medium, which may contain one or more buffer salts (such as sodium bicarbonate), by agglomeration of the pH-adjusting agent into nutritive medium in a fluid bed apparatus, by spray-drying the pH-adjusting agent onto the powdered or agglomerated nutritive medium, or by a combination thereof; this approach obviates the subsequent addition of a pH-adjusting agent after reconstitution of the powdered medium. Thus, the nutritive culture medium described herein is useful in cultivation or growth of cells in vitro that, upon reconstitution with a solvent (e.g., water or serum), has a pH that is optimal for the support of cell cultivation or growth without a need for adjustment of the pH of the liquid medium. This type of medium, defined herein as “automatically pH-adjusting medium,” therefore obviates the time-consuming and error-prone steps of adding buffer(s) to the medium after reconstitution and adjusting the pH of the medium after dissolution of the buffer(s). For example, a mammalian cell culture medium prepared according to these methods may, upon reconstitution, have a pH of between about 7.1 to about 7.5, more preferably between about 7.1 to about 7.4, and most preferably about 7.2 to about 7.4 or about 7.2 to about 7.5.

In another embodiment, automatically pH-adjusting media can be produced by preparing reconstituted media without the addition of any buffering systems or pH-adjusting agents. In a preferred such aspect, an auto-pH medium may be provided by adjusting the buffering systems present in the medium. For example, as one of ordinary skill is aware, culture media typically contain buffers or buffering systems. By adjusting the pH-opposing forms of such buffers in the medium, an auto-pH medium is created, avoiding the requirement to add additional buffers or pH-adjusting agents to achieve a proper pH level prior to or upon reconstitution of the medium and prior to use. In one such aspect described herein, pH-opposing forms of certain media components (particularly phosphate or other buffer salts) are then used in the culture medium to provide a desired pH upon reconstitution of the powdered media. pH-opposing forms of components are conjugate acid-base pairs in which the members of the pair can either raise the pH or lower it to achieve the desired pH of the solution. Sodium HEPES (pH raising) and HEPES-HCl (pH lowering) are examples of pH opposing components. For example, if a reconstituted media having a pH of between 4.5 and 7.2 is to be prepared, the first step is to determine the correct balance of monobasic (to lower the pH) to dibasic (to raise the pH) phosphate in order to yield the desired pH. Typically, mono- and di-basic phosphate salts are used at concentrations of about 0.1 mM to about 10 mM, about 0.2 mM to about 9 mM, about 0.3 mM to about 8.5 mM, about 0.4 mM to about 8 mM, about 0.5 mM to about 7.5 mM, about 0.6 mM to about 7 mM, or preferably about 0.7 mM to about 7 mM. If other buffer systems are used in the formulations, the proper ratio or balance of the basic (typically sodium or monobasic) buffer salt and the corresponding acidic (or pH-opposing; typically, HCl or dibasic) buffer salt is similarly determined to ensure that the formulation will be at the desired final pH upon reconstitution with a solvent. Because the actual phosphate molecular species that is present in a solution is the same at a given pH whether the basic (e.g., sodium or monobasic) or acidic (e.g., HCl or dibasic) form is added, this adjustment would not be expected to impact buffering capacity. Once an appropriate ratio of pH-opposing forms of an appropriate buffer is determined, these components may be added to the medium (for example, a dry powder medium) to provide a culture medium that is of the appropriate pH level upon reconstitution and prior to use.

In one embodiment, the media, media supplement, media subgroup, or buffer dissolves within about 10-30 minutes in water at 25° C. In one aspect, the time until complete dissolution of a medium (e.g., full dissolution rate or t₁₀₀) is within about 1 min, about 2.5 min, about 5 min, about 10 min, about 15 min, about 20 min, about 25 min, or about 30 min. In another aspect, the full dissolution rate is within about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, or about 40 min.

In another embodiment, about 50% of the media, media supplement, media subgroup, or buffer dissolves within about 10-30 minutes in water at 25° C. In one aspect, the time until 50% dissolution of a medium (e.g., t₅₀) is within about 1 min, about 2.5 min, about 5 min, about 10 min, about 15 min, about 20 min, about 25 min, or about 30 min. In another aspect, 50% dissolution rate is within about 2 min, about 3 min, about 4 min, about 5 min, about 6 min, about 7 min, about 8 min, about 9 min, about 10 min, about 11 min, about 12 min, about 13 min, about 14 min, about 15 min, about 16 min, about 17 min, about 18 min, about 19 min, about 20 min, about 21 min, about 22 min, about 23 min, about 24 min, about 25 min, about 26 min, about 27 min, about 28 min, about 29 min, about 30 min, about 31 min, about 32 min, about 33 min, about 34 min, about 35 min, about 36 min, about 37 min, about 38 min, about 39 min, or about 40 min.

Another embodiment is complete dry powder culture media formulations that support the cultivation of cells in vitro upon reconstitution of the media with a solvent, without the need for the addition of any supplemental nutrient components to the medium prior to use. Media according to this aspect described herein thus will preferably comprise the nutritional components necessary for cultivation of a cell in vitro, such that no additional nutritional components need be included in the solvent or added to the medium upon reconstitution and prior to use. Accordingly, such complete media described herein will be suitable for use in cultivating cells in vitro upon reconstitution with water or with an alternative non-nutrient-containing solvent such as a buffered saline solution. Such complete media may be automatically pH-adjusting media, and may comprise one or more culture medium supplements (including but not limited to serum), one or more amino acids (including but not limited to L-glutamine), insulin, transferrin, one or more hormones, one or more lipids, one or more growth factors, one or more cytokines, one or more neurotransmitters, one or more extracts of animal tissues, organs or glands, one or more enzymes, one or more proteins, one or more trace elements, one or more extracellular matrix components, one or more antibiotics, one or more viral inhibitors, one or more buffers, or combinations thereof.

Examples of media supplements that may be prepared by the present methods, or that may be included in the culture media described herein, include, without limitation, animal sera, such as bovine sera, fetal bovine, newborn calf and calf sera, human sera, equine sera, porcine sera, monkey sera, ape sera, rat sera, murine sera, rabbit sera, ovine sera and the like, defined replacements such as StemPro LipoMAX, OptiMAb, Knock-Out Serum Replacement (Gibco, Thermo Fisher Scientific Inc.), hormones (including steroid hormones such as corticosteroids, estrogens, androgens (e.g., testosterone) and peptide hormones such as insulin, cytokines (including growth factors (e.g., EGF, αFGF, βFGF, HGF, IGF-1, IGF-2, NGF and the like), interleukins, colony-stimulating factors, interferons and the like), neurotransmitters, lipids (including phospholipids, sphingolipids, fatty acids, Excyte, cholesterol and the like), attachment factors (including extracellular matrix components such as fibronectin, vitronectin, laminins, collagens, proteoglycans, glycosaminoglycans and the like), and extracts or hydrolysates of animal, tissues (e.g., plant or bacteria tissues), cells, organs or glands (such as bovine pituitary extract, bovine brain extract, chick embryo extract, bovine embryo extract, chicken meat extract, chicken tissue extract, Achilles tendon and extracts thereof) and the like. Other media supplements that may be produced by the present methods or that may be included in the culture media described herein include a variety of proteins (such as serum albumins, particularly bovine or human serum albumins; immunoglobulins and fragments or complexes thereof; aprotinin; hemoglobin; haemin or haematin; enzymes (such as trypsin, collagenases, pancreatinin, or dispase); lipoproteins; fetuin; ferritin; etc.), which may be natural or recombinant; vitamins; amino acids and variants thereof (including, but not limited to, L-glutamine and L-cysteine), enzyme co-factors; polysaccharides; salts or ions (including trace elements such as salts or ions of molybdenum, vanadium, cobalt, manganese, selenium, and the like); and other supplements and compositions that are useful in cultivating cells in vitro that will be familiar to one of ordinary skill. Media supplements produced by the methods described herein include animal or mammalian (e.g., human, fish, bovine, porcine, equine, monkey, ape, rat, murine, rabbit, ovine, insect, etc.) derived supplements, ingredients, or products. These sera and other media supplements are available commercially. Alternatively, sera and other media supplements described herein may be isolated from their natural sources or produced recombinantly by art-known methods that will be routine to one of ordinary skill. See Freshney, R. I., Culture of Animal Cells, New York: Alan R. Liss, Inc., pp. 74-78 (1983), and references cited therein; see also Harlow, E., and Lane, D., Antibodies: A Laboratory Manual, Cold Spring Harbor Laboratory, N.Y, 116-120 (1988).

Components that are often present in the final formulation in μg/mL or even μg/L amounts have typically been left out of standard powdered media due to homogeneity and/or stability concerns, and instead are typically added to the reconstituted 1× media as a concentrate, thereby increasing storage costs and causing production of a finished culture medium to become more costly and less efficient. In one aspect, such low-level components may be added to the base powder by first making a concentrate of the components and then spraying them into a portion of the powdered media that would be granulated with the concentrate. This would then be milled to a particle size in the same general size range as that of the bulk for blending. The ability to spray-in components in small amounts may be especially helpful in developing media that include trace elements, vitamins, viral inhibitors, growth factors, cytokines, and the like. Specifically, among others, the components to be added to a powdered medium include but are not limited to vitamins comprising retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, para-aminobenzoic acid, salts thereof, and trace elements including iron, manganese, copper, iodine, zinc, cobalt, fluoride, chromium, molybdenum, selenium, nickel, silicon, vanadium, salts thereof, or combinations thereof. Additional components to be added in low amounts to the culture media described herein may include, for example, growth factors (e.g., EGF, αFGF, βFGF, KGF, HGF, IGF-1, IGF-2, NGF, insulin, and the like), interleukins, colony-stimulating factors, interferons, attachment factors, extracellular matrix components (e.g., collagens, laminins, proteoglycans, glycosaminoglycans, fibronectin, vitronectin, and the like), lipids (such as phospholipids, cholesterol, bovine cholesterol concentrate, fatty acids, sphingolipids and the like); extracts of animal tissues, glands or organs; antibiotics such as Geneticin carbenicillin, cefotaxime, anti-PPLO, Fungizone, hygromycin, kanamycin, neomycin, nystatin, penicillin, or streptomycin, etc.; and viral inhibitors (e.g., protease inhibitors, nucleoside analogues, and the like, which are known in the art).

Examples of buffers that may be prepared as described herein and/or that may be included in the culture media include, but are not limited to, buffered saline solutions, phosphate-buffered saline (PBS) formulations, Tris-buffered saline (TBS) formulations, HEPES-buffered saline (HBS) formulations, Hanks' Balanced Salt Solutions (HBSS), Dulbecco's PBS (DPBS), Earle's Balanced Salt Solutions, Puck's Saline Solutions, Murashige and Skoog Plant Basal Salt Solutions, Keller's Marine Plant Basal Salt Solutions, Provasoli's Marine Plant Basal Salt Solutions, and Kao and Michayluk's Basal Salt Solutions, and the like. Formulations for these buffers, which are commercially available, as well as for many other commonly used buffers, are well-known in the art and may be found for example in the Thermo Fisher Scientific Catalog, in the DIFCO & BBL Manual, 2ⁿ ed. (Becton, Dickinson and Company, 2009), and in the Millipore Sigma Cell Culture Catalogues.

Clinical solutions, particularly those used for parenteral nutrition, electrolyte balance or intravenous (IV) solutions are also described. Such clinical solutions include but are not limited to Ringer's, Ringer's lactate, 5% by mass dextrose in water, normal saline (0.9% by mass NaCl), hypotonic saline (0.45% by mass NaCl), 5% by mass dextrose in saline, and the like. Clinical solutions may further comprise one or more pharmaceutical compositions or components thereof.

### Methods for Preparing Compositions and Formulations

Also described is a method for preparing nutritive media, media supplements, media subgroups, buffers, or samples that contain a desired or effective amount or concentration of an ingredient(s), wherein at least one ingredient such as a carbohydrate or sugar (e.g., glucose) vitamins, an amino acid, a salt, a trace element, a growth factor and/or an amine (e.g., ethanolamine, spermine, spermidine, putrescene or para-aminobenzoic acid) is inputted into the manufacturing process at a higher amount as compared to the final product. The method of compensating for a loss or decrease in effective concentration of at least one ingredient during an agglomeration process comprising calculating or determining the amount of the ingredient to be added to the process as described herein to result in the final desired or effective amount.

### Methods for Determining the Effective Concentration of a Compound

One method for determining the effective concentration of a compound (e.g., a vitamin) in a test culture medium is as follows. Using a vitamin for the purposes of illustration, a known concentration of the vitamin is serially diluted into a culture medium lacking the vitamin. A second set of serial dilutions are set-up where the test culture medium is serially diluted into a culture medium also lacking the vitamin. Cells that require the vitamin for growth are then added to both sets of serially diluted samples and cultured under appropriate conditions. After a period of time, cell replication is measured (e.g., by cell counting or by measuring optical density). The measurements of the known concentrations are graphed to form a standard curve, to which the measurements from the test culture medium dilutions are compared to determine the effective concentration of the vitamin in the test culture medium. Any number of similar assays may be used to determine the amount of a metabolite(s) in a sample, which are available for cellular metabolism.

### Methods for Sterilizing Compositions and Formulations

Another embodiment is a method for sterilizing the nutritive media, media supplements, media subgroups and buffers described herein, as well as for sterilizing powdered nutritive media, media supplements, media subgroups and buffers prepared by standard methods such as ball-milling. Also described are methods for sterilizing or substantially sterilizing the samples including nutritive media, media supplements, media subgroups, and buffers described herein. Such additional methods may include filtration, heat sterilization, irradiation, or other chemical or physical methods. Nutritive media, media supplements, media subgroups, or buffers (prepared as described herein may be irradiated under conditions favoring sterilization. Since nutritive media, media supplements, media subgroups, and buffers are usually prepared in large volume solutions and frequently contain heat labile components, they are not amenable to sterilization by irradiation or by heating. Thus, nutritive media, media supplements, media subgroups, and buffers are commonly sterilized by contaminant-removal methods such as filtration which significantly increases the expense and time required to manufacture such media, media supplements, media subgroups, and buffers.

Nutritive media, media supplements, media subgroups, or buffers prepared according to the methods described herein can be sterilized by less expensive and more efficient methods. For example, powdered nutritive media, media supplements, media subgroups, or buffers may be irradiated under conditions favoring sterilization of these powders. Preferably, this irradiation is accomplished in bulk (i.e., following packaging of the sample, nutritive media, media supplement, media subgroup, or buffer), and most preferably this irradiation is accomplished by exposure of the bulk packaged sample, media, media supplement, media subgroup, or buffer described herein to a source of gamma rays under conditions such that bacteria, fungi, spores or viruses that may be resident in the powdered sample media, media supplements, media subgroups, or buffers are inactivated (i.e., prevented from replicating). Alternatively, irradiation may be accomplished by exposure of the sample, powdered media, media supplement, media subgroup, or buffer, prior to packaging, to a source of gamma rays or a source of ultraviolet light. The sample, media, media supplements, media subgroups and buffers described herein may alternatively be sterilized by heat treatment (if the subgroups, or components of the sample, nutritive media, media supplement, media subgroup, or buffer are heat stable), for example by flash pasteurization or autoclaving. As will be understood by one of ordinary skill in the art, the dose of irradiation or heat, and the time of exposure, required for sterilization will depend upon the bulk of the materials to be sterilized, and can easily be determined by the ordinarily skilled artisan without undue experimentation using art-known techniques, such as those described herein.

In one embodiment described herein, the bulk sample (e.g., nutritive media, media supplements, media subgroups, or buffers) (which are preferably in powdered form) are exposed to a source of irradiation (e.g., y (gamma) radiation) at a total dosage of about 10-100 kilograys (kGy), preferably a total dosage of about 15-75 kGy, 15-50 kGy, 15-40 kGy, 20-40 kGy or 25-45 kGy, more preferably a total dosage of about 20-30 kGy, and most preferably a total dosage of about 25-35 kGy, for about 1 hour to about 7 days, more preferably about 1 hour to about 5 days, 1 hour to about 3 days, about 1-24 hours or about 1-5 hours, and most preferably about 1-3 hours (“normal dose rate”). Alternatively, the bulk powders described herein may be sterilized at a “slow dose rate” of a total cumulative dosage of about 25-100 kGy over a period of about 1-5 days. During irradiation, the nutritive media, media supplements, media subgroups, or buffers (which are preferably in powdered form) are preferably stored at a temperature of about −70° C. to about room temperature (about 20-25° C.), most preferably at about −70° C. One of ordinary skill will appreciate, of course, that radiation dose and exposure times may be adjusted depending upon the bulk and/or mass of material to be irradiated; typical optimal irradiation dosages, exposure times and storage temperatures required for sterilization of powdered materials by irradiation or heat treatment are known in the art.

Following sterilization, unpackaged nutritive media, media supplements, media subgroups and buffers may be packaged under aseptic conditions, for example by packaging into containers such as sterile tubes, vials, bottles, bags, pouches, boxes, cartons, drums, and the like, in vacuum packaging, or integrated powder/solvent packaging described herein. Sterile packaged samples such as media, media supplements, media subgroups, and buffers may then be stored for extended periods of time as described herein. In one embodiment, eukaryotic cell culture media, feed or supplement compositions are sterilized, freeze-dried and then packaged under aseptic conditions, for example, into containers such as sterile tubes, vials, bottles, bags, pouches, boxes, cartons, drums, and the like.

### Methods for Reconstituting Compositions and Formulations

The nutritive media, media supplements, media subgroups, and buffers described herein are readily soluble in a rehydrating solvent and are substantially dust free. For use, the media, media supplement, media subgroup, or buffer may be “rehydrated” or “reconstituted” in a volume of a solvent sufficient to produce the desired nutrient, electrolyte, ionic and pH conditions required for the particular use of the solvated media, media supplement, media subgroup, or buffer. This reconstitution is particularly facilitated because the media, media supplements, media subgroups, and buffers will readily dissolve and will produce little if any dust or insoluble material, unlike powdered nutritive media, media supplements, media subgroups, or buffers.

Solvents for use in reconstituting the nutritive media, media supplements, media subgroups, buffers, or samples described herein include, but are not limited to, the solvents described herein such as water, such as distilled and/or deionized water, serum (bovine or human serum and most particularly fetal bovine serum or calf serum), organic solvents (dimethylsulfoxide, acetone, ethanol and the like), or any combination thereof, any of which may contain one or more additional components (e.g., salts, polysaccharides, ions, detergents, stabilizers, etc.). For example, media supplements (such as animal sera) and buffers are preferably reconstituted in water to a 1× final concentration, or optionally to a higher concentration (e.g., 2×, 2.5×, 5×, 10×, 20×, 25×, 50×, 100×, 500×, 1000×, etc.) for the preparation of stock solutions or for storage. Alternatively, culture media may be reconstituted in a solution of media supplements (e.g., sera such as FBS) in water, such as those solutions wherein the media supplement is present at a concentration, for example, of 0.5%, 1%, 2%, 2.5%, 5%, 7.5%, 10%, 15%, 20%, 25%, 50%, or higher, by volume (vol/vol) in the water.

Reconstitution of the sample (e.g., nutritive media, media supplements, media subgroups, or buffers) is typically accomplished under aseptic conditions to maintain the sterility of the reconstituted sample, although the reconstituted sample may be sterilized by filtration or other sterilization methods that are well known in the art, following rehydration. Following their reconstitution, media, media supplements, media subgroups and buffers or other samples should be stored at temperatures below about 10° C., preferably at about 1-4° C., until use.

The reconstituted nutritive media, media supplements, media subgroups and buffers may be used to culture or manipulate cells according to standard cell culture techniques that are known to one of ordinary skill in the art. In such techniques, the cells to be cultured are contacted with the reconstituted media, media supplement, media subgroup, or buffer described herein under conditions favoring the cultivation or manipulation of the cells (such as controlled temperature, humidity, lighting, and atmospheric conditions). Cells that are particularly amenable to cultivation by such methods include, but are not limited to, bacterial cells, fish cells, yeast cells, plant cells, and animal cells. Such bacterial cells, yeast cells, plant cells and animal cells are available commercially from known culture depositories, e.g., the American Type Culture Collection (Manassas, Va.) and others that will be familiar to one of ordinary skill in the art. Preferred animal cells for cultivation by these methods include, but are not limited to, insect cells (most preferably Drosophila cells, Spodoptera cells and Trichoplusia cells), nematode cells (most preferably C. elegans cells) and mammalian cells (most preferably CHO cells, COS cells, VERO cells, BHK cells, AE-1 cells, SP2/0 cells, L5.1 cells, hybridoma cells and human cells, such as 293 cells, PER-C6 cells and Hela cells), any of which may be a somatic cell, a germ cell, a normal cell, a diseased cell, a transformed cell, a mutant cell, a stem cell, a precursor cell or an embryonic cell, embryonic stem cells (ES cells), cells used for virus or vector production (i.e., 293, PerC 6), cells derived from primary human sites used for cell or gene therapy, i.e., lymphocytes, hematopoietic cells, other white blood cells (WBC), macrophage, neutrophils, dendritic cells, and any of which may be an anchorage-dependent or anchorage-independent (i.e., “suspension”) cell. Another aspect is the manipulation or cultivation of cells and/or tissues for tissue or organ transplantation or engineering, i.e., hepatocyte, pancreatic islets, osteoblasts, osteoclasts/chondrocytes, dermal or muscle or other connective tissue, epithelial cells, tissues like keratinocytes, cells of neural origin, cornea, skin, organs, and cells used as vaccines, i.e., blood cells, hematopoietic cells other stem cells or progenitor cells, and inactivated or modified tumor cells of various histotypes.

### Methods for Culturing or Manipulating Cells

Another embodiment is a method for culturing or manipulating one or more cells comprising contacting said cells with the cell culture reagents described herein, particularly reconstituted nutritive media, media supplement, media subgroup, or buffer and incubating said cell or cells under conditions favoring the cultivation or manipulation of the cell or cells. Any cell may be cultured or manipulated according to the present methods, particularly bacterial cells, yeast cells, plant cells, animal cells and other cells or cell lines described herein. Cells cultured or manipulated according to this aspect described herein may be normal cells, diseased cells, transformed cells, mutant cells, somatic cells, germ cells, stem cells, precursor cells or embryonic cells, any of which may be established cell lines or obtained from natural sources.

Nutritive media, media supplements and media subgroups produced by the present methods are any media, media supplement or media subgroup (serum-free or serum-containing) which may be used to manipulate or support the growth of a cell, which may be a bacterial cell, a fungal cell (particularly a yeast cell), a plant cell, or an animal cell (particularly an insect cell, a nematode cell, or a mammalian cell, most preferably a human cell), any of which may be a somatic cell, a germ cell, a normal cell, a diseased cell, a transformed cell, a mutant cell, a stem cell, a precursor cell, or an embryonic cell. Preferred such nutritive media include, but are not limited to, cell culture media, most preferably a bacterial cell culture medium, plant cell culture medium or animal cell culture medium. Preferred media supplements include, but are not limited to, undefined supplements such as extracts or hydrolysates of bacterial, animal or plant cells, glands, tissues or organs (particularly bovine pituitary extract, bovine brain extract and chick embryo extract); and biological fluids or blood derived products (particularly animal sera, and most preferably bovine serum (particularly fetal bovine, newborn calf, or normal calf serum), horse serum, porcine serum, rat serum, murine serum, rabbit serum, monkey serum, ape serum, or human serum, any of which may be fetal serum) and extracts thereof (more preferably serum albumin and most preferably bovine serum albumin or human serum albumin). Medium supplements may also include defined replacements such as StemPro LipoMAX, OptiMAb, Knock-Out Serum Replacement (Gibco, Thermo Fisher Scientific Inc.), and the like, which can be used as substitutes for the undefined media supplements described above. Such supplements may also comprise defined components, including but not limited to, hormones, cytokines, neurotransmitters, lipids, attachment factors, proteins, amino acids, and the like.

The media described herein, upon being reconstituted with a solvent, can be used for the growth and/or cultivation of organisms such as, e.g., filamentous fungi, transgenic plants (e.g., tobacco, rice, and Lemna), lichens, or algae, or cells derived from any of the aforementioned organisms.

### Forms of Media, Supplements, or Feeds

Also described herein are forms of media, supplements, or feeds. In one aspect, the supplement includes one or more amino acids. In another aspect, a salt of an amino acid is used. In another aspect, the salt is a sodium salt. In another aspect, monobasic and dibasic phosphate salts are used. A preferred cation is sodium. In another aspect, the monobasic and dibasic salts are provided such that a resultant pH, for example, about 8 pH is obtained. Depending on the formulation, while the ratio of monobasic to dibasic salts may be dictated by desired pH, different total salt concentrations should be tried to optimize solubility, especially when concentrated or highly concentrated supplements are to be used. The pH can also be confirmed when assessing the salt concentration. When an amino acid is not provided as a salt, preferably the pH effect of the acid is countered by a tribasic phosphate, preferably a sodium tribasic phosphate. While sodium is preferred as a cation, other metals, such as potassium, calcium, magnesium may be used. If a specific counter ion is desired, it may be available as a phosphate salt. In another aspect, the supplement powder dissolves rapidly. In another aspect, the supplement can be prepared and used as a highly concentrated mixture, for example, with one or more components at a concentration about 2× or more, preferably 3×, 5×, 8×, 10×, 12×, 15×, 20×, 25×, 50×, 75×, 85×, 95×, or even about 100× or more times the concentration of that component in the medium being supplemented. The concentration of each desired ingredient of the supplement can be independently selected. In another aspect, the supplement is prepared by reconstituting with water under sterile conditions. In another aspect, the supplement is sterilized by filtration.

A supplement may have no ingredients in common with the medium being supplemented or may have one or more ingredients in common. The supplement may differ from the medium being supplemented in at least one manner, such as a different concentration of one or more ingredients, for example a different ratio of two ingredients, a different ingredient mix, additional ingredients, or omitted ingredients in the supplement. For example, a supplement may omit salts to the extent feasible and may contain, for example, significantly enhanced concentrations of growth factors or amino acids. A preferred supplement formulation contains at least 2, more preferably 3, but perhaps at least 4, 5, 6, 7, 8, 9, 10, 11, 12, 13, 14, 15, 17, 20, or more amino acids including salts, or dimers thereof.

In some embodiments, feed supplements as described herein are utilized to supplement a medium that has or is being used to culture cells, e.g., as the cells are cultured, some ingredients are removed from the medium by the cells. In some embodiments described herein, the feed supplement is used, inter alia, to replace some or all of these ingredients. In some embodiments, the supplement contains the majority of the ingredients that were in the original medium to be supplemented, but the feed medium is lacking at least one ingredient. In some embodiments, the feed supplement is lacking 1, 2, 3, 4, 5, 6, 7, 8, 9, 10, 11, 12, 13, 14, 15, or more ingredients as compared to the concentration in the original culture medium being supplemented. In some embodiments, the feed supplement is added in a concentrated form, e.g., at 2×, 3×, 4×, 5×, 6×, 7×, 8×, 9×, 10×, 15×, 20×, 30×, 40×, 50×, 100×, 200×, 300×, 400×, 500× or 1000×. By concentrated form is meant that at least one of the ingredients in the feed supplement is at a concentration higher than what is the desired concentration in the culture medium. In some embodiments, ingredients for a feed supplement may be divided into multiple feed supplement media, e.g., based upon compatible subgroups.

In another embodiment, a media, supplement, feed, subgroup, or buffer is added to a culture to optimize the concentration of a particular component, supplement a depleted or omitted component, or replace a component that has been consumed or degraded by the culture. The media can be dissolved in a solvent prior to addition to the culture or added in solid form.

Chemically defined cell culture media are formulated to support optimal cell growth and function while minimizing variability. Typical components in these media include amino acids (such as glutamine, arginine, and cysteine), water-soluble vitamins (e.g., vitamin B12 and ascorbic acid), trace elements (e.g., zinc, copper, selenium, and manganese), carbohydrates (e.g., glucose), lipids and fatty acids, buffering agents (e.g., sodium bicarbonate), growth factors, and inorganic salts (e.g., sodium, potassium, calcium, and magnesium chlorides).

To confirm cell culture media stability, both Ultra-Performance Liquid Chromatography (UPLC) and High-Performance Liquid Chromatography (HPLC) can be used to ensure accurate quantitation of liable media components following the lyophilization process. Quantification and confirmation of preserved amino acids, water soluble vitamins, fatty acids and carbon sources are critical to confirm media stability. Effectiveness of less liable components such as trance metals and buffers can be confirmed via cell performance assays.

In another embodiment, a cell culture is analyzed to determine the concentration of one or more media components. Typical analysis methods can be used to determine the concentration of a component such as HPLC, mass spectrometry, ELISA, standard curve assays, and other methods known in the art. If the concentration of a component is less than a desired range, a supplement may be added to the culture to increase the concentration to the desired range. The media can be dissolved in a solvent prior to addition to the culture or added and dissolved in situ.

In some embodiments, a portion of the ingredients of a feed supplement is reconstituted from an agglomerated supplement. In some embodiments, additional ingredients are added to the reconstituted media or a liquid form of a supplement or feed. In some embodiments, the additional ingredients comprise amino acids or antibiotics.

### Osmolality of Compositions or Formulations

Osmolality (a measure of osmotic pressure) of cell culture medium is important as it helps regulate the flow of substances in and out of the cell. It is typically controlled by the addition or subtraction of salt in a culture medium. Rapid increases in osmolality (e.g., addition of concentrated feed supplement with elevated osmolality relative to the base growth medium) may result in stressed, damaged, or dead cells. Maintaining an optimal osmolality range during cell culture/growth is desirable for cell function and/or bioproduction success.

Liquid base growth medium typically has osmolality ranges of about 200 mOsm/kg to about 400 mOsm/kg. Concentrated feed supplements have an osmolality of about 1000 mOsm/kg to about 3000 mOsm/kg. In some embodiments, the osmolality of a base medium has an osmolality of about 100 mOsm/kg to about 500 mOsm/kg, about 100 to about 400 mOsm/kg, about 100 mOsm/kg to about 300 mOsm/kg, about 100 mOsm/kg to about 200 mOsm/kg, 150 mOsm/kg to about 500 mOsm/kg, about 150 to about 400 mOsm/kg, about 150 mOsm/kg to about 300 mOsm/kg, about 150 mOsm/kg to about 200 mOsm/kg, about 200 mOsm/kg to about 500 mOsm/kg, about 200 mOsm/kg to about 400 mOsm/kg, about 200 mOsm/kg to about 300 mOsm/kg, about 200 mOsm/kg to about 250 mOsm/kg, about 250 mOsm/kg to about 400 mOsm/kg, about 300 mOsm/kg to about 400 mOsm/kg, about 350 mOsm/kg to about mOsm/kg, about 400 mOsm/kg to about 500 mOsm/kg, or about 450 mOsm/kg to about 500 mOsm/kg.

In some embodiments, the osmolality of a concentrated feed supplement medium described herein (e.g., a 5× concentrated feed supplement medium) has an osmolality between from about 1000 mOsm/kg to about 4000 mOsm/kg about, about 1000 mOsm/kg to about 3500 mOsm/kg, about 1000 mOsm/kg to about 3000 mOsm/kg, about 1000 mOsm/kg to about 2500 mOsm/kg, about 1000 mOsm/kg to about 2000 mOsm/kg, about 2000 mOsm/kg to about 3500 mOsm/kg, about 2000 mOsm/kg to about 3000 mOsm/kg, about 2000 mOsm/kg to about 2500 mOsm/kg, about 3000 mOsm/kg to about 3500 mOsm/kg, about 3000 mOsm/kg to about 4000 mOsm/kg, about 1000 mOsm/kg, about 1500 mOsm/kg, about 2000 mOsm/kg, about 2500 mOsm/kg, about 3000 mOsm/kg, about 3500 mOsm/kg, or about 4000 mOsm/kg.

In some embodiments, the osmolality of a concentrated feed supplement medium described herein is between about 4× to about 10× as compared to the osmolality of the medium being supplemented or fed. In some aspects, the osmolality of a concentrated feed supplement medium described herein is between about 3.0× to about 3.5×, about 3.5× to about 4.5×, about 4.5× to about 5.5×, about 5.5× to about 6.5×, about 6.5× to about 7.5×, about 7.5× to about 8.5×, about 8.5× to about 9.5×, about 9.5× to about 10.5×, about 10.5× to about 11.5×, about 11.5× to about 12.5×, about 12.5× to about 13.5×, about 13.5× to about 14.5×, about 14.5× to 18.5 to about 19.5×, about 19.5× to about 20.5×, about 3× to about 10×, about 5× to about 10×, about 10× to about 15×, about 15× to about 20×, about 20× to about 25×, or about 25× to about 100× as compared to the osmolality of the medium being supplemented or fed.

In some embodiments, addition of a concentrated feed supplement described herein increases the osmolality of a medium by about 25 mOsm/kg to about 100 mOsm/kg or by about 0 to about 100, about 0.01 to about 100, about 0.1 to about 100, about 1 to about 100, about 10 to about 100, about 50 to about 100, about 75 to about 100, about 1 to about 10, about 1 to about 50, about 1 to about 75, about 10 to about 50, about 15 to about 35, about 25 to about 50, or about 20 to about 30 mOsm/kg.

### Methods for Preparing Media at a Reduced Cost

Described herein are methods for the preparation of nutritive media, media supplements, media subgroups, buffers, and cells at reduced cost and time (e.g., better efficiency). The compositions and methods provide for the preparation of nutritive media, media supplements, media subgroups, buffers, and cells at reduced cost and reduced time and inconvenience. The cost reductions are due to the several factors. For example, the media, media supplement, media subgroup, and buffer formulations may be produced with smaller production facilities since the large stir tanks required for 1× formulations are not required. In addition, the media, media supplement, media subgroup and buffer formulations may be prepared on an as needed basis using “just in time” production techniques, which reduce inventory, storage, and labor costs. The time required for the preparation and shipping of the media, media supplement, media subgroup and buffer formulations may be reduced from 6-8 weeks to as little as one day. The automatically pH-adjusting media described herein also provide significant cost, time savings, and reduce the tendency for introduction of contamination into reconstituted media that may occur during the pH adjustment process according to methods using traditional dry powder or bulk liquid media.

### Preparation of Large Quantities of a Composition and Formulation

Also described is the preparation of nutritive media, media supplements, media subgroups, or buffers which may be used to prepare very large quantities of 1× media, media supplements, media subgroups, or buffers (e.g., 100,000 liters or more) which would require only one quality control test compared to multiple quality control tests for multiple batches produced according to other commonly used techniques. Importantly, the media, media supplement, media subgroup, or buffer formulations are more consistent between batches since the individual components are more stable. Further, the media, media supplement, media subgroup, or buffer formulations can easily be dispensed to produce a particular volume without having to weigh the dry component.

### Methods for Producing Agglomerated Powders

Another embodiment described herein is a method for producing a form of an agglomerated nutritive medium powder, an agglomerated medium supplement powder, an agglomerated nutritive medium subgroup powder, or an agglomerated buffer powder, said method comprising agglomerating a nutritive medium powder, medium supplement powder, nutritive medium subgroup powder, or buffer powder, with a solvent comprising at least one lipid dissolved therein, said solvent delivering said at least one lipid for incorporation in said nutritive medium powder, medium supplement powder, nutritive medium subgroup powder, or buffer powder. In one aspect, the agglomerating comprises fluid bed agglomeration.

### Kits

The media, media supplements, media subgroups, buffers, cells, and cell-containing compositions described herein are ideally suited for preparation of kits. Such a kit may comprise one or more containers such as vials, test tubes, bottles, packages, pouches, drums, lyophilization bags and/or containers, and the like. Each of the containers may contain one or more of the cell culture reagents, nutritive media, media supplements, media subgroups, cells, or buffers described herein, or combinations thereof. Such cell culture reagents, nutritive media, media supplements, media subgroups, buffers or cells may be hydrated or dehydrated but are typically dehydrated preparations produced by the methods described herein. Such preparations may be sterile or substantially sterile.

A container may contain, for example, a nutritive media, media supplement, media subgroup, or a buffer described herein, or any component or subgroup thereof, such as any of those nutritive media, media supplements, media subgroups, or buffers that are described herein. Additional dry, or liquid nutritive media, buffers, extracts, supplements, components, or subgroups may be contained in additional containers in the present kits. The kits may also contain, in one or more additional containers, one or more cells such as bacterial cells, yeast cells, plant cells, or animal cells. Such cells may be lyophilized, dried, frozen, or otherwise preserved, or may be spray-dried according to the methods described herein or treated by the method described herein. In addition, the kits described herein may further comprise one or more additional containers, containing, for example, L-glutamine, optionally complexed with one or more divalent cations. The kits may further comprise one or more additional containers containing a solvent to be used in reconstituting the dry powder pharmaceutical, or clinical compositions, cell culture reagents, nutritive media, media supplements, media subgroups and/or buffers; such solvents may be aqueous or organic and include buffer solutions, saline solutions, nutritive medium solutions, nutritive medium supplement solutions including sera such as bovine sera, fetal bovine sera, calf sera, human sera, or combinations thereof. Other ingredients that are not compatible for admixture with the nutritive media, buffers, pharmaceutical compositions, extracts, supplements, components, or subgroups described herein may be contained in one or more additional containers to avoid mixing of incompatible components. An exemplary kit may comprise a container containing media or reconstitution optionally of a volume sufficient to contain the reconstituting solvent, instructions for reconstitution and means for accessing the media such as a tear strip or a port for introducing the reconstituting solvent.

The number and types of containers contained in a given kit (e.g., for making a nutritive medium, medium supplement, medium subgroup, or buffer) may vary depending on the desired product or the type of pharmaceutical or clinical compositions, media, media supplement, media subgroup, or buffer to be prepared. Typically, the kit will contain the respective containers containing the components or supplements necessary to make a particular pharmaceutical or clinical composition, media, media supplement, media subgroup, or buffer. However, additional containers may be included in the kit described herein so that different pharmaceutical or clinical compositions, media, media supplements, media subgroups, or buffers can be prepared by mixing different amounts of various components, supplements, subgroups, buffers, solvents, etc., to make different pharmaceutical or clinical compositions, media, media supplement, media subgroup, or buffer formulations. The containers may be sealable, re-sealable, tamper-resistant, tamper evident, or open-indicating. The containers may have multiple modes, such as a resealable bag within a tamper-resistant or open-evident sealed pail.

Kits may also contain one or more of labels, manufacturing dates, expiration dates, use by dates, packaging, branding, instructions for use, material safety data sheets (MSDS), hazard or warning placards, or product literature.

### EMBODIMENTS

One embodiment described herein is a freeze-dried eukaryotic cell culture media, feed or supplement composition having enhanced stability. In one aspect, the composition is stable at a temperature of about 1° C. to about 40° C. In another aspect, the composition is stable at a temperature of about 15° C. to about 37° C. In another aspect, the composition is stable at refrigeration temperature for about 1 day to about 5 years. In another aspect, the composition is stable at room temperature for about 1 day to about 2 years. In another aspect, the composition is subjected to a freezing temperature ranging from about −60° C. to about −5° C. for about 4 hours to about 10 hours. In another aspect, the composition is subjected to a freezing temperature ranging from about −60° C. to about −40° C. for about 4 hours to about 10 hours. In another aspect, the composition is subjected to a drying temperature ranging from about 30° C. to about 70° C. for about 1 hour to about 4 hours. In another aspect, the composition is subjected to a drying temperature ranging from about 40° C. to about 60° C. for about 1 hour to about 4 hours. In another aspect, the composition has a reduced moisture content as compared to a non-freeze-dried cell culture medium. In another aspect, the composition has a moisture content of at least 1% by mass less than the moisture content of a non-freeze-dried cell culture medium. In another aspect, the composition has a moisture content of about 2-2.6% by mass. In another aspect, the composition is a dry powder media composition. In another aspect, the composition is an agglomerated composition. In another aspect, the composition is animal-origin free. In another aspect, the composition is chemically-defined. In another aspect, the composition is protein-free. In another aspect, the composition is serum-free. In another aspect, the composition has an osmolality of about 200 mOsm/kg to about 3000 mOsm/kg upon reconstitution. In another aspect, the composition has a pH of about 6.9 to about 7.5 upon reconstitution. In another aspect, the composition comprises: one or more amino acids, salts, buffers, trace minerals, lipids, nucleic acids, or proteins; one or more carbohydrates; and one or more vitamins. In another aspect, the carbohydrates comprise one or more of glucose, fructose, or trehalose. In another aspect, the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.

Another embodiment described herein is a kit comprising a freeze-dried eukaryotic cell culture media, feed, or supplement composition, and one or more containers comprising drums, buckets, boxes, bags, pouches, or combinations thereof. In one aspect, the kit optionally further comprises one or more of labels, manufacturing dates, expiration dates, use by dates, packaging, branding, instructions for use, material safety data sheets (MSDS), hazard or warning placards, or product literature.

Another embodiment described herein is a method for freeze-drying a cell culture media, feed, or supplement composition to enhance stability, the method comprising: subjecting the composition to a freezing temperature ranging from about −60° C. to about −5° C.; and subjecting the composition to a drying temperature ranging from about 30° C. to about 70° C.

Another embodiment described herein is a freeze-dried cell culture media, feed, or supplement composition produced by any of the methods described herein.

Another embodiment described herein is a method for stabilizing a cell culture media, feed, or supplement composition by freeze-drying, the method comprising: providing a cell culture media, feed, or supplement composition or component thereof; subjecting the composition or component thereof to a freezing temperature ranging from about −60° C. to about −5° C.; subjecting the composition or component thereof to a drying temperature ranging from about 30° C. to about 70° C.; and packaging the composition or component thereof in a container.

Another embodiment described herein is the use of the freeze-dried cell culture media, feed, or supplement compositions described herein, or the kits described herein for cell culture. In one aspect, the composition is used for culturing a mammalian cell in vitro. In another aspect, the mammalian cell comprises CHO cells or HEK cells.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### CLAUSES

Various embodiments and aspects of the inventions described herein are summarized by the following clauses:
Clause 1. A freeze-dried eukaryotic cell culture media, feed or supplement composition having enhanced stability.
Clause 2. The composition of clause 1, wherein the composition is stable at a temperature of about 1° C. to about 40° C.
Clause 3. The composition of clause 1 or 2, wherein the composition is stable at a temperature of about 15° C. to about 37° C.
Clause 4. The composition of any one of clauses 1-3, wherein the composition is stable at refrigeration temperature for about 1 day to about 5 years.
Clause 5. The composition of any one of clauses 1-4, wherein the composition is stable at room temperature for about 1 day to about 2 years.
Clause 6. The composition of any one of clauses 1-5, wherein the composition is subjected to a freezing temperature ranging from about −60° C. to about −5° C. for about 4 hours to about 10 hours.
Clause 7. The composition of any one of clauses 1-6, wherein the composition is subjected to a freezing temperature ranging from about −60° C. to about −40° C. for about 4 hours to about 10 hours.
Clause 8. The composition of any one of clauses 1-7, wherein the composition is subjected to a drying temperature ranging from about 30° C. to about 70° C. for about 1 hour to about 4 hours.
Clause 9. The composition of any one of clauses 1-8, wherein the composition is subjected to a drying temperature ranging from about 40° C. to about 60° C. for about 1 hour to about 4 hours.
Clause 10. The composition of any one of clauses 1-9, wherein the composition has a reduced moisture content as compared to a non-freeze-dried cell culture medium.
Clause 11. The composition of any one of clauses 1-10, wherein the composition has a moisture content of at least1% by mass less than the moisture content of a non-freeze-dried cell culture medium.
Clause 12. The composition of any one of clauses 1-11, wherein the composition has a moisture content of about 2-2.6% by mass.
Clause 13. The composition of any one of clauses 1-12, wherein the composition is a dry powder media composition.
Clause 14. The composition of any one of clauses 1-13, wherein the composition is an agglomerated composition.
Clause 15. The composition of any one of clauses 1-14, wherein the composition is animal-origin free.
Clause 16. The composition of any one of clauses 1-15, wherein the composition is chemically-defined.
Clause 17. The composition of any one of clauses 1-16, wherein the composition is protein-free.
Clause 18. The composition of any one of clauses 1-17, wherein the composition is serum-free.
Clause 19. The composition of any one of clauses 1-18, wherein the composition has an osmolality of about 200 mOsm/kg to about 3000 mOsm/kg upon reconstitution.
Clause 20. The composition of any one of clauses 1-19, wherein the composition has a pH of about 6.9 to about 7.5 upon reconstitution.
Clause 21. The composition of any one of clauses 1-20, wherein the composition comprises:
one or more amino acids, salts, buffers, trace minerals, lipids, nucleic acids, or proteins;
one or more carbohydrates; and
one or more vitamins.
Clause 22. The composition of any one of clauses 1-21, wherein the carbohydrates comprise one or more of glucose, fructose, or trehalose.
Clause 23. The composition of any one of clauses 1-21, wherein the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.
Clause 24. A kit comprising the composition of any one of clauses 1-23, and one or more containers comprising drums, buckets, boxes, bags, pouches, or combinations thereof.
Clause 25. The kit of clause 24, wherein the kit optionally further comprises one or more of labels, manufacturing dates, expiration dates, use by dates, packaging, branding, instructions for use, material safety data sheets (MSDS), hazard or warning placards, or product literature.
Clause 26. A method for freeze-drying a cell culture media, feed, or supplement composition to enhance stability, the method comprising:
subjecting the composition to a freezing temperature ranging from about −60° C. to about −5° C.; and
subjecting the composition to a drying temperature ranging from about 30° C. to about 70° C.
Clause 27. A freeze-dried cell culture media, feed, or supplement composition produced by the method of clause 26.
Clause 28. A method for stabilizing a cell culture media, feed, or supplement composition by freeze-drying, the method comprising:
providing a cell culture media, feed, or supplement composition or component thereof;
subjecting the composition or component thereof to a freezing temperature ranging from about −60° C. to about −5° C.;
subjecting the composition or component thereof to a drying temperature ranging from about 30° C. to about 70° C.; and
packaging the composition or component thereof in a container.
Clause 29. Use of the freeze-dried cell culture media, feed, or supplement compositions of any one of clauses 1-23, or the kit of clause 24 or 25 for cell culture.
Clause 30. The use of clause 29, wherein the composition is used for culturing a mammalian cell in vitro.
Clause 31. The use of clause 30, wherein the mammalian cell comprises CHO cells or HEK cells.

### EXAMPLES

### Example 1

### Freeze-Dried Cell Culture Formulations

Experiments were designed to evaluate the use of freeze-drying on the stability and shelf-life of GIBCO DYNAMIS AGT media (Catalog Number A2617501).

Cell culture media powder requires careful storage to ensure that the integrity of all components is conserved. Through the process of freeze-drying, the shelf-life of dry powder media can be increased by reducing the water content to significantly lower amounts allowing for preservation of sensitive components due to the reduced presence of water molecules.

Freeze-drying of AGT media is a technology that was developed to reduce water content, improve the shelf-life, and improve room temperature stability of the disclosed media products. A key advantage of this freeze-drying technology is that freeze-drying dry agglomerated media allows further reduction of moisture content and improved product stability. The advantage provided is the extended stability of sensitive components that would otherwise interact with and breakdown from the presence of water.

When water content is reduced, less reactivity of sensitive compounds is observed. This freeze-drying technology may be applied to create room temperature stable media and media that is able to be stored at refrigerated temperatures for a greater duration of time. Further, room temperature stability reduces cost throughout the logistical process following the manufacturing of dry agglomerated media. By freeze-drying, more stable media can be achieved that is able to be stored at room temperature for a period of time or stored for longer times in refrigeration.

The process for freeze-drying dry agglomerated media involves placing the powder onto a tray, setting the parameters of the freeze-dryer, inserting the dry agglomerated media into the freeze-dryer, and running the cycle. The freeze-drying process reduces the water content of the already low-moisture dry agglomerated media and prevents the interaction of sensitive compounds and water reducing degradation of the media. This reduced water content allows for the dry agglomerated media to be preserved for greater periods of time without any change in the components of the media.

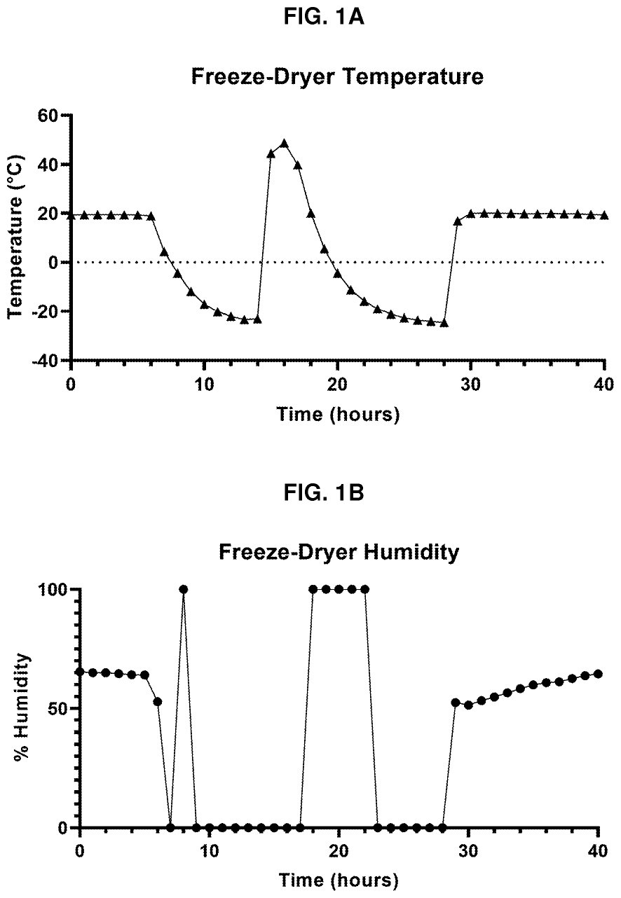

One non-limiting exemplary process for freeze-drying dry agglomerated media that was utilized included a drying temperature of 51° C. and an initial freezing temperature of −23° C. This experimental approach was set up by placing pre-weighed cups of DYNAMIS AGT media onto the tray of a HARVEST RIGHT at-home freeze-dryer where initial weights of the AGT media were recorded, and the built-in freeze-drying cycle was then run overnight. See FIG. 1A-B.

Chemically defined cell culture media are formulated to support optimal cell growth and function while minimizing variability. Typical components in these media include amino acids (such as glutamine, arginine, and cysteine), water-soluble vitamins (e.g., vitamin B12 and ascorbic acid), trace elements (e.g., zinc, copper, selenium, and manganese), carbohydrates (e.g., glucose), lipids and fatty acids, buffering agents (e.g., sodium bicarbonate), growth factors, and inorganic salts (e.g., sodium, potassium, calcium, and magnesium chlorides).

To confirm cell culture media stability, we employed both Ultra-Performance Liquid Chromatography (UPLC) and High-Performance Liquid Chromatography (HPLC) to ensure accurate quantitation of liable media components following the lyophilization process. Quantification and confirmation of preserved amino acids, water soluble vitamins, fatty acids and carbon sources are critical to confirm media stability. Effectiveness of less liable components such as trance metals and buffers were confirmed via cell performance assays.

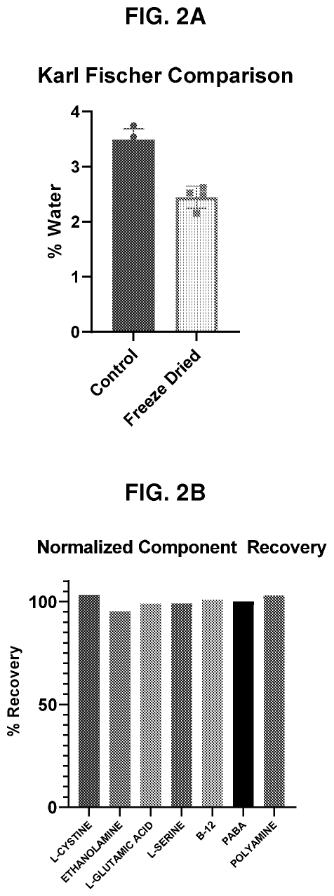

When the AGT media was removed from the freeze-dryer, it was immediately weighed again to gauge the loss of mass. This loss of mass was found to be an average decrease of 0.77% of the final weight. To verify that this decrease in weight was lost water, the freeze-dried samples, along with non-freeze dried DYNAMIS AGT control samples from the same lot, were submitted for analysis by titration. The results are shown in FIG. 2A. The data of FIG. 2A show that the percentage of final product mass that is water was reduced by 1.03% from 3.48% in the control condition samples down to 2.45% in the freeze-dried condition samples.

On the same day that the samples were removed from the freeze-dryer, they were reconstituted and submitted for analysis. One freeze-dried sample and a control sample from the same lot were submitted and the results showed no substantial degradation of components due to the freeze-drying process. Data from freeze dry samples were normalized to control (non-freeze-dried) samples which were set to 100% for all components, including L-cystine, ethanolamine, L-glutamic acid, L-serine, B-12, PABA and polyamine. The normalized data from the analytical chemistry results are shown in FIG. 2B. Samples were analyzed in triplicate, error bars are SEM.

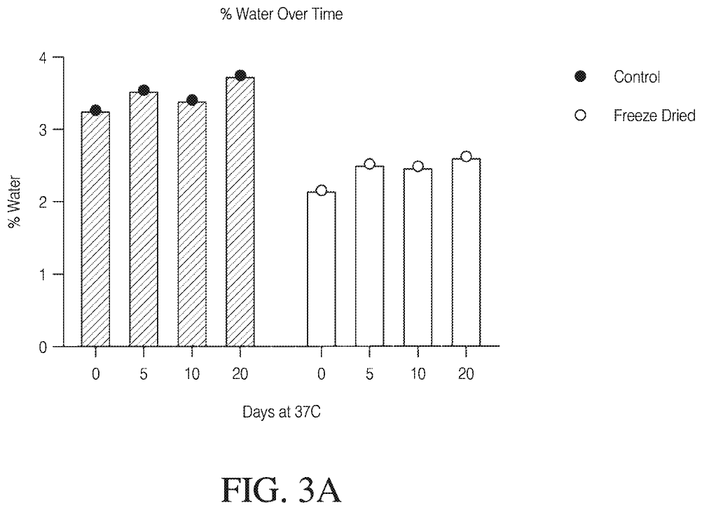

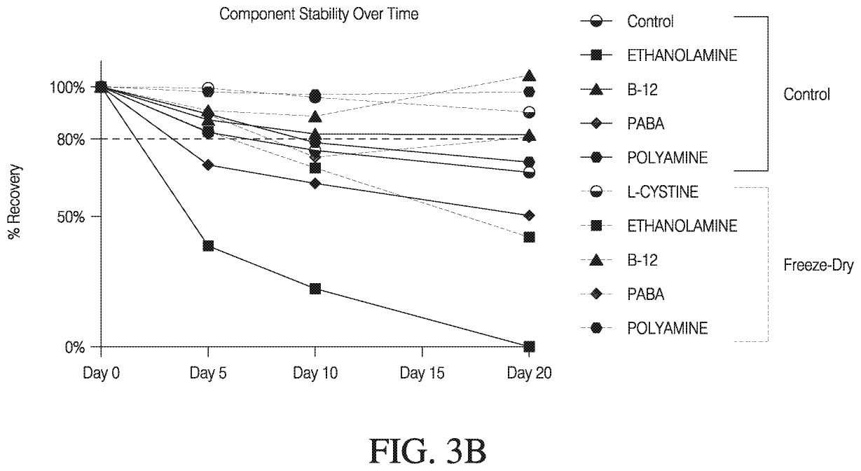

Once it was confirmed that the various components were not destroyed by freeze-drying (FIG. 2B), an accelerated shelf-life study was performed which involved placing the samples into a 37° C. environmental chamber for 20 days total. This was carried out by removing a sample after 5, 10, and 20 days, and performing analytical chemistry analyses on the samples after reconstituting them on the day of the timepoint. To ensure that the shelf-life study was as accurate as possible, 3 freeze-dried samples and 3 control samples from the same lot were packaged in the same heat-sealed poly(ethylene vinyl alcohol) (EVOH) bags at the same time. The results are shown in FIG. 3A. The control is the first 4 bars on FIG. 3A and freeze-dried is the second 4 bars on FIG. 3B. This shelf-life study demonstrated that the removal of 1% water by lyophilization successfully extended the shelf-life of DYNAMIS AGT and could be used to extend the shelf-life of other dry agglomerated and powder media.

FIG. 3B specifically shows selected degradation-prone compounds that had the most notable decreases in percent recovery over time, including L-cystine, ethanolamine, B-12, PABA, and polyamine. For example, ethanolamine in the control sample was undetectable by Day 20, but 42% remained in the freeze-dried sample. The data of FIG. 3B show that while degradation was present in both conditions, it was more prevalent in the control samples. Notably, many commercial media contain components such L-cystine, ethanolamine, L-glutamic acid, L-serine, B-12, PABA and polyamine and thus, can be similarly tested and measured for degradation. With the parameters that were tested, it was found that the temperatures used for freeze-drying did not destroy any compounds in the media, and that the process preserved the media when compared to a non-freeze-dried control.

Overall, these data confirmed that freeze-drying is capable of increasing the shelf-life of DYNAMIS AGT and potentially other dry agglomerated and powder media. This can be tested by measuring the degradation rate of sensitive components in the media (supra).

For the tests described above, one methodology, one freeze-dryer, and one cycle type were used for freeze-drying. Optionally, a larger-scale freeze-dryer with greater customizability may be used so that the specific parameters used could be more finely adjusted. For example, some different parameters that could be modulated include the cycle length, the initial freeze temperature, subsequence freezing steps, and temperatures, the drying temperature(s), the number of cycles, and the specific freeze-dryer/lyophilizer that is used. The adjustment of any of these parameters may produce similar or even superior results. For instance, an industrial freeze-dryer intended for use pharmaceuticals may remove a greater amount of water and further improve the results. In the experiments described above, the water content was able to be reduced by about 1% down to a final water content of 2.5%. Further studies may be able to reduce the final water content down to 1%, or even lower.

### Example 2

### Quantitation of Labile Media Components

To assess cell culture media, the inventors have used both Ultra-Performance Liquid Chromatography (UPLC) and High-Performance Liquid Chromatography (HPLC) to ensure accurate quantitation of labile media components. UPLC was utilized to quantification amino acids including alanine, glycine, phenylalanine, histidine, proline, arginine, hydroxyproline, serine, asparagine, isoleucine, ethanolamine, aspartic acid, leucine, threonine, glutamic acid, lysine HCl, tyrosine, glutamine, methionine, valine, and tryptophan. HPCL and UPLC techniques were used to quantify water-soluble vitamins such as cyanocobalamin, folic acid, niacinamide, riboflavin, and biotin.

The complete list of components tested are shown below:
Amino Acids
L-ALANINE
L-ARGININE
L-ASPARAGINE H2O
L-ASPARTIC ACID
L-CYSTINE
ETHANOLAMINE HCl
L-GLUTAMINE
L-GLUTAMIC ACID
GLYCINE
L-HISTIDINE
HYDROXY-L-PROLINE
L-ISOLEUCINE
L-LEUCINE
L-LYSINE HCl
L-METHIONINE
AMMONIA
L-PHENYLALANINE
L-PROLINE
L-SERINE
L-THREONINE
L-TRYPTOPHAN
L-TYROSINE
L-VALINE
Vitamins
B-12
FOLIC ACID
NIACINAMIDE
PHENOL RED
RIBOFLAVIN
THIAMINE HCl
L-TRYPTOPHAN

### Example 3

### Testing Freeze-Drying for Room Temperature Stable DYNAMIS AGT Media

Further experiments are designed and conducted to evaluate if freeze-drying can produce room temperature stable DYNAMIS AGT.

These experiments may include a larger-scale freeze-dryer with greater customizability so that specific parameters may be more finely adjusted to reduce the water content. For example, some different parameters that may be modulated include the cycle length, initial freeze temperature, final drying temperature, number of cycles, and the specific freeze-dryer/lyophilizer that is used.

Freeze-drying using optimal parameters will result in further reduction of the total water content to enhance stability and shelf-life, ultimately generating room temperature stable DYNAMIS AGT.

It is appreciated that the freeze-drying processes described herein may be applied to multiple cell culture media formulations beyond DYNAMIS AGT, including EFC+ formulations and glucose-free variants of agglomerated media. It was observed experimentally that glucose-free formulations exhibited a greater reduction in residual moisture content following freeze-drying as compared to corresponding glucose-containing formulations. This effect is attributed to the hygroscopic nature of glucose, which may retain water within the matrix and limit the extent of moisture removal during lyophilization. In contrast, glucose-free formulations demonstrated enhanced drying efficiency, resulting in lower final moisture levels. Notably, EFC+ formulations subjected to freeze-drying did not exhibit any significant decrease in the abundance of measured components, indicating that the freeze-drying process can be broadly applicable across different media compositions while maintaining component integrity.

In some aspect, freeze-drying improves the stability of multiple degradation-prone components present in cell culture media during storage at elevated temperature. In an accelerated stability study conducted at 37° C. over a period of 20 days, control (non-freeze-dried) samples exhibited significant decreases in the concentrations of para-aminobenzoic acid (PABA), L-cystine, ethanolamine, and putrescine. Corresponding freeze-dried samples demonstrated improved retention of these components over the same time period. For example, ethanolamine levels decreased to undetectable levels in control samples by day 20, whereas measurable quantities remained in freeze-dried samples. Similarly, PABA, L-cystine, and putrescine were better preserved in freeze-dried samples relative to controls. These results indicate that freeze-drying reduces degradation across multiple classes of labile components, including amino acids, vitamins, and polyamines.

In other aspects, analytical characterization of freeze-dried and control media includes evaluation of multiple classes of components, including amino acids, vitamins, and polyamines. Representative analytes include, but are not limited to, amino acids such as L-cystine and ethanolamine, vitamins such as cobalamin (vitamin B12), riboflavin, and para-aminobenzoic acid (PABA), and polyamines such as putrescine. Analysis of these components following freeze-drying and during storage demonstrates that freeze-dried formulations exhibit improved stability across these component classes relative to non-freeze-dried controls. In certain embodiments, the majority of analyzed components show minimal loss in concentration following freeze-drying and enhanced retention during storage under accelerated conditions.

The stability of freeze-dried media may be further characterized by monitoring moisture content over time under elevated temperature conditions. When stored in a sealed environment at 37° C., freeze-dried samples maintain lower moisture content relative to non-freeze-dried controls throughout the duration of the study. While a slight increase in moisture content may be observed during early timepoints, for example due to handling or packaging, the overall moisture levels in freeze-dried samples remain significantly reduced compared to control samples over extended storage periods. This sustained reduction in moisture content is associated with improved preservation of labile components and reduced degradation reactions.

It is appreciated that the effects of freeze-drying on media stability can be qualitatively assessed through visual inspection following storage. After storage at elevated temperature, for example at 37° C. for up to 20 days, non-freeze-dried control samples may exhibit visible discoloration or browning, indicative of chemical degradation processes. In contrast, freeze-dried samples stored under the same conditions exhibit reduced or minimal discoloration, maintaining a more consistent visual appearance relative to initial conditions. This reduction in browning is consistent with decreased degradation of media components in freeze-dried formulations.

In some aspects, while freeze-drying significantly improves the stability of cell culture media, certain components may still undergo partial degradation during storage. For example, ethanolamine, a known labile component, may decrease in concentration over time even in freeze-dried samples, although to a lesser extent than in non-freeze-dried controls. These observations indicate that freeze-drying reduces, but may not eliminate, degradation of all components. Accordingly, further optimization of formulations and/or processing conditions may be employed in combination with freeze-drying to further enhance stability.

The extent of moisture removal achieved during freeze-drying may depend on the composition of the media formulation. In particular, the presence of hygroscopic components such as glucose may contribute to retention of residual moisture within the matrix, thereby limiting the efficiency of water removal during lyophilization. Formulations lacking such hygroscopic components may exhibit greater reductions in moisture content under otherwise similar freeze-drying conditions. These compositional effects may be considered when optimizing formulations for enhanced stability.

1. A freeze-dried eukaryotic cell culture media, feed or supplement composition having enhanced stability.

2. The composition of claim 1, wherein the composition is stable at a temperature of about 1° C. to about 40° C.

3. The composition of claim 2, wherein the composition is stable at a temperature of about 15° C. to about 37° C.

4. The composition of claim 1, wherein the composition is stable at refrigeration temperature for about 1 day to about 5 years.

5. (canceled)

6. The composition of claim 1, wherein the composition is subjected to a freezing temperature ranging from about −60° C. to about −5° C. for about 4 hours to about 10 hours.

7. The composition of claim 1, wherein the composition is subjected to a freezing temperature ranging from about −60° C. to about −40° C. for about 4 hours to about 10 hours.

8. The composition of claim 1, wherein the composition is subjected to a drying temperature ranging from about 30° C. to about 70° C. for about 1 hour to about 4 hours.

9. The composition of claim 1, wherein the composition is subjected to a drying temperature ranging from about 40° C. to about 60° C. for about 1 hour to about 4 hours.

10. The composition of claim 1, wherein the composition has a reduced moisture content as compared to a non-freeze-dried cell culture medium.

11. The composition of claim 1, wherein the composition has a moisture content of at least 1% by mass less than the moisture content of a non-freeze-dried cell culture medium.

12. The composition of claim 1, wherein the composition has a moisture content of about 2-2.6% by mass.

13. The composition of claim 1, wherein the composition is a dry powder media composition, an agglomerated composition, animal-origin free, chemically-defined, protein-free, and/or protein-free.

14. (canceled)

15. (canceled)

16. (canceled)

17. (canceled)

18. (canceled)

19. The composition of claim 1, wherein the composition has an osmolality of about 200 mOsm/kg to about 3000 mOsm/kg upon reconstitution.

20. The composition of claim 1, wherein the composition has a pH of about 6.9 to about 7.5 upon reconstitution.

21. The composition of claim 1, wherein the composition comprises:
one or more amino acids, salts, buffers, trace minerals, lipids, nucleic acids, or proteins;
one or more carbohydrates; and
one or more vitamins.

22. The composition of claim 21, wherein the carbohydrates comprise one or more of glucose, fructose, or trehalose.

23. The composition of claim 21, wherein the one or more vitamins comprise one or more of retinol (A), thiamine (B1), riboflavin (B2), niacinamide (B3), pantothenic acid (B5), pyridoxamine (B6), biotin (B7), folic acid (B9) cobalamin (B12), ascorbic acid (C), cholecalciferol (D), tocopherol (E), phylloquinone (K), choline, inositol, lipoic acid, or para-aminobenzoic acid.

24. A kit comprising the composition of claim 1, and one or more containers comprising drums, buckets, boxes, bags, pouches, or combinations thereof.

25. (canceled)

26. (canceled)

27. (canceled)

28. A method for stabilizing a eukaryotic cell culture media, feed, or supplement composition by freeze-drying, the method comprising:
providing a cell culture media, feed, or supplement composition or component thereof;
subjecting the composition or component thereof to a freezing temperature ranging from about −60° C. to about −5° C.;
subjecting the composition or component thereof to a drying temperature ranging from about 30° C. to about 70° C.; and
packaging the composition or component thereof in a container.

29. (canceled)

30. (canceled)

31. (canceled)

32. The composition of claim 1, wherein the composition is a mammalian cell culture media, feed, or supplement composition.
